(19) United States
(12) Patent Application Publication
(10) Pub. No.: US 2009/0198241 A1
(43) Pub. Date: Aug. 6, 2009

(54) SPINE DISTRACTION TOOLS AND METHODS OF USE

(76) Inventor: Christopher U. Phan, San Leandro, CA (US)

Correspondence Address: MEDTRONIC;Attn: Noreen Johnson - IP Legal Department, 2600 Sofamor Danek Drive, Memphis, TN, 38132, US

(21) Appl. No.: 12/182,431

(22) Filed: Jul. 30, 2008

Related U.S. Application Data
(60) Provisional application No. 61/025,991, filed on Feb. 4, 2008.

(51) Int. Cl. A61B 17/58; A61F 5/00

(52) U.S. Cl. 606/90; 606/102; 606/86 R

(57) ABSTRACT

An apparatus includes a measurement tool having a size configured to change when the measurement tool is moved between a first configuration and a second configuration. The measurement tool includes a first spacer member and a second spacer member configured to move relative to each other when the tool is moved between the first and second configurations. The measurement tool has a distal actuator having a first actuator surface matingly and movably coupled to the first spacer member, and a second actuator surface matingly and movably coupled to the second spacer member. A proximal actuator is coupled to a proximal end portion of the elongate member and is configured to rotate about an axis substantially parallel to at least a portion of a center line of an elongate member to move the distal actuator. The distal actuator is configured to move the first spacer member relative to the second spacer.

### CROSS-REFERENCE TO RELATED APPLICATIONS

This application claims priority to U.S. Provisional Application Ser. No. 61/025,991, entitled “Medical Implants and Methods,” filed Feb. 4, 2008, which is incorporated herein by reference in its entirety.

This application is related to U.S. Patent Application Attorney Docket Nos. KYPH-040/01US 305363-2273, entitled “Medical Implants and Methods,” KYPH-040/02US 305363-2271, entitled “Tools and Methods for Insertion and Removal of Medical Implants,” and KYPH-040/03US 305363-2270, entitled “Medical Implants and Methods,” each filed on same date, the disclosures of each are hereby incorporated herein by reference in their entirety.

### BACKGROUND

The invention relates generally to the treatment of spinal conditions, including, for example, the treatment of spinal compression using percutaneous spinal implants for implantation between adjacent spinous processes and/or percutaneous spinal implants for implantation in a space associated with an intervertebral disc.

Minimally-invasive procedures have been developed to provide access to the space between adjacent spinous processes such that major surgery is not required. Such known procedures, however, may not be suitable in conditions where the spinous processes are severely compressed. When the spinous processes are compressed, it can be difficult to insert a spinal implant between adjacent spinous processes. Moreover, such procedures can involve large or multiple incisions. Further, some of the known implants configured to be inserted into a space associated with an intervertebral disc or between adjacent spinous processes may require actuation to an expanded configuration after being inserted into the desired position. Tools for providing such actuation can be difficult to maneuver within the patient's body. Often, multiple tools are required to insert and remove an implant and to actuate an implant after being placed at a desired location.

Thus, a need exists for improvements in the methods and tools used for the insertion and removal of spinal implants, such as implants for implantation between adjacent spinous processes and/or implants for implantation in a space associated with an intervertebral disc. In addition, a need exists for improvements in devices and methods for distracting anatomical structures to provide access for an implant.

### SUMMARY OF THE INVENTION

Medical devices and related methods for the treatment of spinal conditions are described herein. In some embodiments, an apparatus includes a measurement tool coupled to a distal end portion of an elongate member. A size of the measurement tool is configured to change when the measurement tool is moved between a first configuration and a second configuration. The measurement tool includes a spacer having a first spacer member and a second spacer member. The first spacer member is configured to move relative to the second spacer member when the measurement tool is moved between the first configuration and the second configuration. The measurement tool also has a distal actuator that has a first actuator surface that is matingly and movably coupled to the first spacer member, and a second actuator surface that is matingly and movably coupled to the second spacer member. A proximal actuator is coupled to a proximal end portion of the elongate member and is configured to rotate about an axis substantially parallel to at least a portion of a center line of the elongate member to move the distal actuator. The distal actuator is configured to move the first spacer member relative to the second spacer.

### BRIEF DESCRIPTION OF THE DRAWINGS

FIG. 1 is a schematic illustration of an insertion/removal tool according to an embodiment, and an implant shown in a first configuration.

FIGS. 2 and 3 are schematic illustrations of the insertion/removal tool of FIG. 1 shown between a first spinous process and a second spinous process, and the implant of FIG. 1 shown in a first configuration and a second configuration, respectively.

FIGS. 4 and 5 are schematic illustrations of a dilation device according to an embodiment shown in a first configuration and a second configuration, respectively.

FIGS. 6 and 7 are perspective views of a dilation device according to an embodiment shown in a first configuration and a second configuration, respectively.

FIG. 8 is a side perspective view of the dilation head of the dilation device shown in FIG. 7 in the first configuration.

FIG. 9 is a cross-sectional view of the dilation head shown in FIG. 8 in the first configuration, taken along line X-X in FIG. 8.

FIG. 10 is a perspective view of the dilation head of the dilation tool shown in FIG. 7 in the second configuration.

FIG. 11 is a cross-sectional view of the dilation head shown in FIG. 10 in the second configuration.

FIG. 12 is a cross-sectional view of the dilation device shown in FIG. 6 in the first configuration.

FIG. 13 is an enlarged cross-sectional view of the dilation device shown in FIG. 12.

FIG. 14 is a side perspective view of the outer shaft of the dilation device of FIG. 6.

FIG. 15 is a side perspective view of the handle of the dilation device of FIG. 6.

FIG. 16 is a side perspective view of the drive shaft of the dilation device of FIG. 6.

FIG. 17 is a side perspective view of the indicator of the dilation device of FIG. 6.

FIG. 18 is a side perspective view of the lock tab of the dilation device of FIG. 6.

FIG. 19 is a top perspective view of an implant according to an embodiment, in a first configuration.

FIG. 20 is a side perspective view of the implant shown in FIG. 19 in the first configuration.

FIG. 21 is a cross-sectional view of the implant shown in FIGS. 19 and 20, taken along line X-X in FIG. 19.

FIG. 22 is a top perspective view of the implant shown in FIG. 19 in a second configuration.

FIG. 23 is a side perspective view of the implant shown in FIG. 19 in the second configuration.

FIG. 24 is a cross-sectional view of the implant shown in FIGS. 23 and 24 in the second configuration.

FIGS. 25 and 26 are exploded views of the implant illustrated in FIGS. 19-24.

FIG. 27 is a side perspective view of an insertion/removal tool, according to an embodiment.

FIG. 28 is side cross-sectional view of the insertion/removal device of FIG. 27.

FIG. 29 is a side perspective view of the outer shaft of the insertion/removal tool of FIG. 27.

FIG. 30 is a side perspective view of the intermediate shaft of the insertion/removal tool of FIG. 27.

FIG. 31 is a side perspective view of the inner shaft of the insertion/removal tool of FIG. 27.

FIG. 32 is a distal perspective view of a portion of the insertion/removal tool of FIG. 27.

FIG. 33 is an end perspective view of the implant of FIG. 19.

FIG. 34 is an exploded view of a portion of the insertion/removal tool of FIG. 27.

FIG. 35 is a side perspective view of the release knob and housing coupler of the insertion/removal tool of FIG. 27.

FIG. 36 is a perspective cross-sectional view of the release knob and housing coupler of FIG. 35.

FIG. 37 is an exploded view of a portion of the insertion/removal tool of FIG. 27.

FIG. 38 is a side perspective view of the actuation handle and release knob coupler of the insertion/removal tool of FIG. 27.

FIG. 39 is a perspective cross-sectional view of the actuation handle and release knob coupler of FIG. 38.

FIGS. 40 and 41 are perspective views of the insertion/removal tool of FIG. 27 and the implant of FIG. 19 shown in a first configuration and a second configuration, respectively.

FIGS. 42 and 43 are perspective views of an insertion/removal tool according to another embodiment of the invention and an implant according to another embodiment shown in a first configuration and a second configuration, respectively.

FIG. 44 is a side perspective view of an insertion/removal device according to another embodiment and an implant according to another embodiment.

FIG. 45 is a distal perspective view of the insertion/removal tool of FIG. 44

FIG. 46 is a side cross-sectional view of a portion of the insertion/removal tool of FIG. 44 and the implant of FIG. 44.

FIG. 47 is an end perspective view of the implant of FIG. 44.

FIG. 48 is a side cross-sectional view of a portion of the insertion/removal tool of FIG. 44.

FIG. 49 is a side perspective view of a portion of the insertion/removal tool of FIG. 44.

FIG. 50 is a side perspective view of a portion of the intermediate shaft of the insertion/removal tool of FIG. 44.

FIG. 51 is a side perspective view of the outer shaft of the insertion/removal tool of FIG. 44.

FIG. 52 is a side perspective view of the inner shaft of the insertion/removal tool of FIG. 44.

FIG. 53 is a side perspective cross-sectional view of the handle of the insertion/removal tool of FIG. 44.

FIG. 54 is a bottom perspective view of the release knob of the insertion/removal tool of FIG. 44.

### DETAILED DESCRIPTION

Devices and methods for performing medical procedures are described herein. Dilation tools are described that can be used to dilate or distract adjacent anatomical structures, such as adjacent spinous process implants. Such devices can be also be configured to provide an indication or measurement of the amount of distraction. Also described herein are various implant insertion/removal tools and implants. The insertion/removal tools can be used to insert percutaneously an implant into, for example, a space between adjacent spinous processes, or within an intervertebral disc space, and then used to actuate the implant between a first configuration (e.g., collapsed configuration) and a second configuration (e.g., expanded configuration). The insertion/removal tools can also be used to reposition or remove an implant from the patient's body. For example, an insertion/removal tool as described herein can be inserted into the patient's body and coupled to the implant while the implant is still implanted in the body.

In some embodiments, an apparatus includes a first elongate member that defines a lumen and a second elongate member that is movably disposed within the lumen of the first elongate member. A distal end portion of the first elongate member is configured to be releasably coupled to a spinal implant. A distal end portion of the second elongate member includes a driving member configured to engage an actuation member of the spinal implant when the first elongate member is coupled to the spinal implant. The driving member is configured to rotate the actuation member to move the spinal implant between a collapsed configuration and an expanded configuration. The first elongate member configured to secure the spinal implant to the first elongate member.

In some embodiments, a method includes coupling a distal end portion of a first elongate member of an insertion tool to a first coupling portion on a spinal implant such that the spinal implant is prevented from longitudinal movement relative to the insertion tool. A distal end portion of a second elongate member of the insertion tool is inserted into a second coupling portion of the spinal implant such that the distal end portion of the insertion tool engages an actuator of the spinal implant. The second elongate member is movably disposed within a lumen of the first elongate member. The spinal implant is then disposed into a selected location within a patient's body. The second elongate member is then rotated relative to the first elongate member such that the actuator of the spinal implant is rotated and moves the spinal implant from a collapsed configuration to an expanded configuration.

In some embodiments, an apparatus includes a first elongate member that defines a lumen and a second elongate member that is movably disposed within the lumen of the first elongate member. The second elongate member is movably disposed within a lumen of a third elongate member. The first elongate member includes a first coupling portion configured to be coupled to a spinal implant such that the spinal implant is prevented from movement relative to the first elongate member along a longitudinal axis defined by a distal end portion of the first elongate member. The second elongate member includes a second coupling portion configured to be coupled to the spinal implant. The second elongate member is configured to actuate the implant between a first configuration and a second configuration when the second elongate member is rotated relative to the first elongate member.

In one embodiment, an apparatus includes a measurement tool coupled to a distal end portion of an elongate member. A size of the measurement tool is configured to change by a first amount when the measurement tool is moved between a first configuration and a second configuration. An actuator is coupled to a proximal end portion of the elongate member and is configured to rotate about an axis substantially parallel to at least a portion of a center line of the elongate member to move the measurement tool between the first and the second configurations. A size indicator is disposed at a proximal end portion of the elongate member that is configured to move axially relative to the elongate member by a second amount when the measurement tool is moved between the first and second configurations.

In another embodiment, an apparatus includes an elongate member having a center line that is non-linear. The elongate member has a first shaft and a second shaft and at least a portion of the second shaft is movably disposed within first shaft. A measurement tool is coupled to a distal end portion of the elongate member. A size of the measurement tool is configured to change when the measurement tool is moved between a first configuration and a second configuration. An actuator is configured to rotate the second shaft relative to the first shaft to move the measurement tool between the first configuration and the second configuration. A size indicator is configured to indicate the change in the size of the measurement tool when the measurement tool is moved between the first configuration and the second configuration.

In some embodiments, an apparatus includes a measurement tool coupled to a distal end portion of an elongate member. A size of the measurement tool is configured to change when the measurement tool is moved between a first configuration and a second configuration. The measurement tool includes a spacer having a first spacer member and a second spacer member. The first spacer member is configured to move relative to the second spacer member when the measurement tool is moved between the first configuration and the second configuration. The measurement tool also has a distal actuator that has a first actuator surface that is matingly and movably coupled to the first spacer member, and a second actuator surface that is matingly and movably coupled to the second spacer member. A proximal actuator is coupled to a proximal end portion of the elongate member and is configured to rotate about an axis substantially parallel to at least a portion of a center line of the elongate member to move the distal actuator. The distal actuator is configured to move the first spacer member relative to the second spacer.

As used in this specification and the appended claims, the singular forms “a,” “an” and “the” include plural referents unless the context clearly dictates otherwise. Thus, for example, the term “a member” is intended to mean a single member or a combination of members, “a material” is intended to mean one or more materials, or a combination thereof. Furthermore, the words “proximal” and “distal” refer to direction closer to and away from, respectively, an operator (e.g., surgeon, physician, nurse, technician, etc.) who would insert the medical device into the patient, with the tip-end (i.e., distal end) of the device inserted inside a patient's body first. Thus, for example, the implant end first inserted inside the patient's body would be the distal end of the implant, while the implant end to last enter the patient's body would be the proximal end of the implant.

The term “parallel” is used herein to describe a relationship between two geometric constructions (e.g., two lines, two planes, a line and a plane, two curved surfaces, a line and a curved surface or the like) in which the two geometric constructions are substantially non-intersecting as they extend substantially to infinity. For example, as used herein, a line is said to be parallel to a curved surface when the line and the curved surface do not intersect as they extend to infinity. Similarly, when a planar surface (i.e., a two-dimensional surface) is said to be parallel to a line, every point along the line is spaced apart from the nearest portion of the surface by a substantially equal distance. Two geometric constructions are described herein as being “parallel” or “substantially parallel” to each other when they are nominally parallel to each other, such as for example, when they are parallel to each other within a tolerance. Such tolerances can include, for example, manufacturing tolerances, measurement tolerances or the like.

The term “normal” is used herein to describe a relationship between two geometric constructions (e.g., two lines, two planes, a line and a plane, two curved surfaces, a line and a curved surface or the like) in which the two geometric constructions intersect at an angle of approximately 90 degrees within at least one plane. For example, as used herein, a line is said to be normal to a curved surface when the line and an axis tangent to the curved surface intersect at an angle of approximately 90 degrees within a plane. Two geometric constructions are described herein as being “normal” or “substantially normal” to each other when they are nominally normal to each other, such as for example, when they are normal to each other within a tolerance. Such tolerances can include, for example, manufacturing tolerances, measurement tolerances or the like.

It should be understood that the references to geometric constructions are for purposes of discussion and illustration. The actual structures may differ from geometric ideal due to tolerances and/or other minor deviations from the geometric ideal.

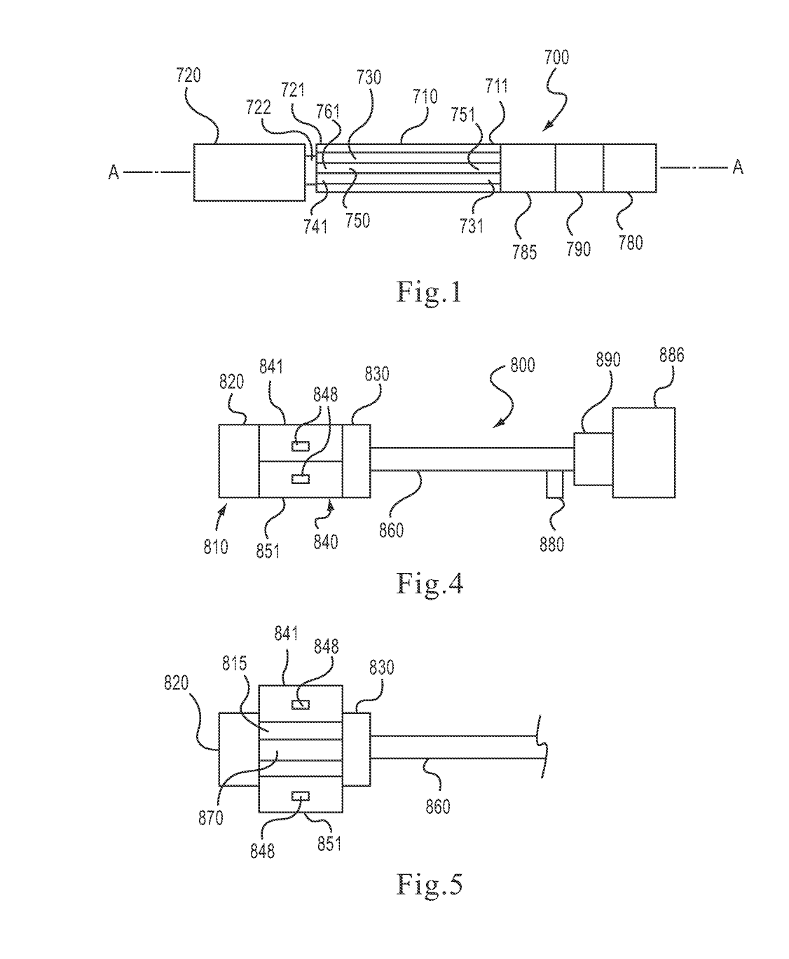

FIG. 1 is a schematic illustration of an insertion/removal tool 700 according to an embodiment of the invention coupled to a spinal implant 720. The insertion/removal tool 700 can include an inner shaft 750 movably disposed within a lumen (not shown) of an intermediate shaft 730, and an outer shaft 710. Although not in cross-section, for illustration purposes, FIG. 1 is a schematic representation of the intermediate shaft 730 and the inner shaft 750 that would otherwise not be visible through the outer shaft 710. The intermediate shaft 730 is movably disposed within a lumen (not shown in FIG. 1) of the outer shaft 710. A proximal end portion 711 of the outer shaft 710 is coupled to a housing 785. A proximal end portion 731 of the intermediate shaft 730 is coupled to a release knob 790 and a proximal end portion 751 of the inner shaft is coupled to a handle 780.

The inner shaft 750, intermediate shaft 730 and outer shaft 710 share a common longitudinal axis A-A. The release knob 790 can be rotated to actuate movement of the intermediate shaft 730, and the handle 780 can be rotated independent of the release knob 790 to actuate movement of the inner shaft 750.

A distal end portion 721 of the outer shaft 710 can include a coupling portion configured to be coupled to, or engage an implant engagement member 722 of an implant 720 as described in more detail below with reference to specific embodiments. For example, in some embodiments, the distal end portion 721 of the outer shaft 710 defines an opening configured to receive an external implant engagement member of the spinal implant. Alternatively, the implant engagement member 722 can have an opening that can receive a portion of the insertion/removal tool 700. In some embodiments, the outer shaft 710 when coupled to the spinal implant can prevent the spinal implant from rotating relative to the insertion/removal tool 700.

A distal end portion 741 of the intermediate shaft 730 can include a coupling portion configured to be coupled to a mating coupling portion of the spinal implant 720. In some embodiments, the distal end portion 741 of the intermediate shaft 730 includes a threaded portion (not shown in FIG. 1) configured to be threadedly coupled to a corresponding threaded portion on the spinal implant 720. In some embodiments, the distal end portion 741 of the intermediate shaft 730 includes a quick connect feature configured to be releasably coupled to a corresponding quick connect feature on the spinal implant 720. The intermediate shaft 730 when coupled to the spinal implant 720 can prevent the spinal implant 720 from moving longitudinally relative to the insertion/removal tool 700.

A distal end portion 761 of the inner shaft 750 can be coupled to the spinal implant 720 and used to actuate the spinal implant 720 between a collapsed configuration and an expanded configuration. For example, the distal end portion 761 of the inner shaft 750 can include a drive portion or member (not shown in FIG. 1) configured to engage a head of a threaded actuating member or drive screw (not shown in FIG. 1) of the spinal implant 720. The drive member can be, for example, a hexagon-shaped protrusion configured to be received within a hexagon-shaped opening of threaded actuating member. The inner shaft 750 can be, for example, spring loaded at its proximal end portion 751 such that the distal end portion 761 is biased distally to ensure the drive member fits tightly into the head of a drive screw (as described in more detail below) of a spinal implant.

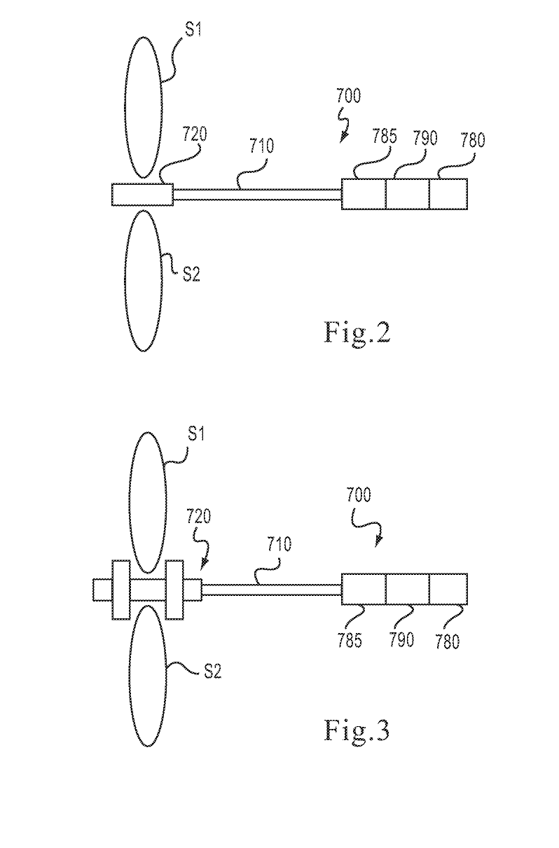

The insertion/removal tool 700 can be used to insert the spinal implant 720 into a desired location within a patient's body and actuate the spinal implant 720 between a collapsed configuration and an expanded configuration. For example, the insertion/removal tool 700 can be coupled to the spinal implant 720 by securing the intermediate shaft 730 to the implant engagement member 722 of spinal implant 720 (as described in more detail below) and coupling the drive member of the inner shaft 750 to the actuating member (drive screw) of the spinal implant 720. With the spinal implant 720 in a collapsed configuration, the insertion/removal tool 700 can then be used to insert percutaneously the spinal implant 720 into a space between adjacent spinous processes S1 and S2 as shown schematically in FIG. 2.

Once positioned at a desired location, the inner shaft 750 can be actuated by rotating the handle 780 independently from the release knob 790 and the housing 785, which in turn causes the actuating member (e.g., drive screw) of the spinal implant 720 to rotate and moves the spinal implant 720 from the collapsed configuration to an expanded configuration as shown in FIG. 3. In this example, the spinal implant 720 when in the expanded configuration is configured to limit lateral movement of the spinal implant 720 when positioned between the adjacent spinous processes S1 and S2. In some embodiments, the spinal implant 720 can be configured to limit extension of the adjacent spinous processes, while allowing for flexion. In other embodiments, the insertion/removal tool 700 can be used to insert and actuate other types of implants, such as for example, an implant configured to be disposed within an intervertebral disc space. Such implants are described in U.S. Patent Application Attorney Docket No. KYPH-040/01US 305363-2277, which is incorporated herein by reference in its entirety.

After the spinal implant 720 has been expanded and is secure within a desired location, the intermediate shaft 730 can be decoupled from the implant engagement portion 722 of the implant 720 via rotation of the release knob 790. The implant insertion/removal tool 700 can then be removed from the body while leaving the spinal implant 720 in position within the body of a patient.

The implant insertion/removal tool 700 can also be used to remove and/or reposition an implant already disposed within the body of a patient. For example, the insertion/removal tool 700 can be coupled to the spinal implant 720 while the spinal implant 720 is disposed within the patient's body in the same manner as described above. The spinal implant 720 can then be moved to its collapsed configuration by rotating the actuation handle 780 of the insertion/removal tool 700 in an opposite direction such that the drive member rotates the actuating member of the spinal implant 720 and moves the spinal implant 720 to the collapsed configuration. With the spinal implant 720 secured to the insertion/removal tool 700, the insertion/removal tool 700 can be used to move or reposition the spinal implant 720 within the patient's body, or remove the spinal implant 720 from the patient's body.

FIGS. 4 and 5 are each a schematic illustration of a dilation device (also referred to herein as a “distraction device”) according to an embodiment. A dilation device 800 can be used to distract adjacent anatomical structures, such as adjacent spinous processes. The distraction device 800 can also be used to dilate or distract tissue within a patient's body. In some embodiments, a dilation device 800 can be used to measure a distance between adjacent anatomical structures.

The dilation device 800 can include a dilation head 810, an outer shaft 860, a drive shaft 870 (shown in FIG. 5) movably disposed within a lumen (not shown in FIG. 4) of the outer shaft 860, a lock tab 880, a handle 886 and an indicator 890. The dilation head 810 has a distal end portion 820, a proximal end portion 830 and a central portion 840. The dilation head 810 also defines a lumen (not shown in FIG. 4).

The central portion 840 includes a first dilation member 841 and a second dilation member 851. The first dilation member 841 and the second dilation member 851 are each configured to be moved between a first configuration as shown in FIG. 4 and a second configuration as shown in FIG. 5. For example, the drive shaft 870 is coupled to the distal end portion 820 of the dilation head 810 and is used to move the distal end portion 820 and the proximal end portion 830 to and from each other as described in more detail below with reference to a specific embodiment.

The central portion 840 of the dilation head 810 can also include one or more markers 848 that be used to position the dilation head 810 at a desired location within a patient's body. For example, the markers 848 can be radiotranslucent holes that are viewable on a fluoroscope.

The handle 886 of the dilation tool 800 is coupled to the drive shaft 870 and to the indicator 890. The handle 886 of the dilation tool 800 is configured to rotate the drive shaft 870 of the dilation tool 1300 when in the second configuration. The lock tab 880 of the dilation tool 800 is configured to engage the outer shaft 860 (described in more detail below) to prevent the handle 886 from rotating with respect to the outer shaft 860. The indicator 890 of the dilation tool 800 can be used to determine the amount of dilation produced by expanding the first dilation member 841 and the second dilation member 851. For example, the indicator 890 can move axially along the outer shaft 860, and the amount of axial movement traveled by the indicator 890 can correspond to the amount of distraction made by the dilation device 800.

In use, dilation head 810 of the dilation tool 800 while in the first configuration (FIG. 4) is inserted percutaneously between adjacent anatomical structures, such as in a space between a pair of adjacent spinous processes. The distal end portion 820 of the dilation head 810 is inserted first and is moved until the central portion 840 is positioned between the anatomical structures. Once in a desired location, the dilation tool 800 can be moved from the first configuration (FIG. 4) to the second configuration (FIG. 5). As the dilation tool 800 is moved to the second configuration, the first dilation member 841 and the second dilation member 851 contact the adjacent anatomical structures and exert a force to dilate or distract the adjacent anatomical structures. The amount of distraction can be observed on the indicator 890. After distracting the anatomical structures a desired amount, the dilation tool 800 can be moved back to the first configuration (FIG. 4) to remove the dilation tool 800 from the patient's body.

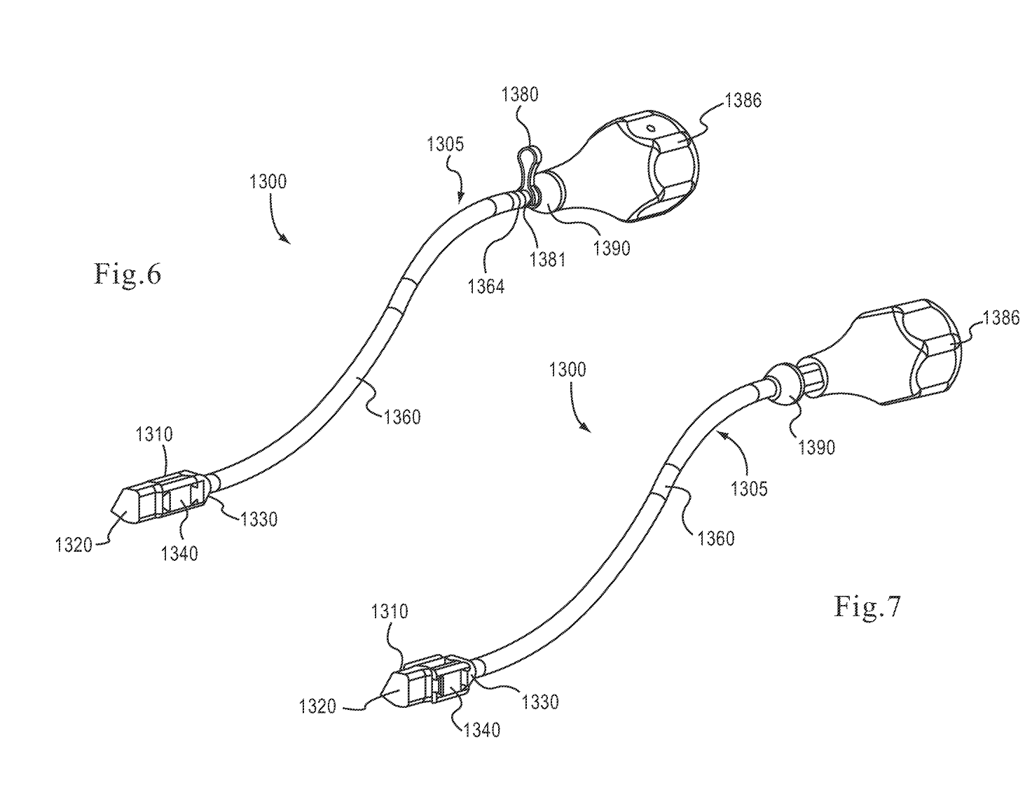

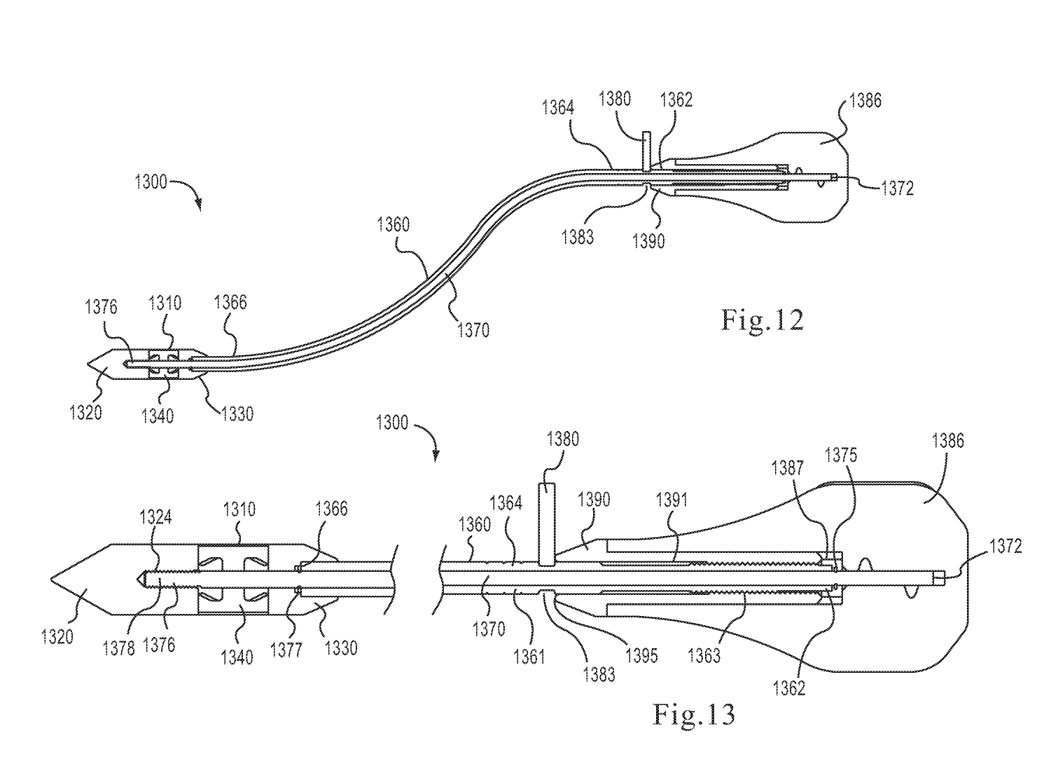

FIGS. 6-18 illustrate a dilation tool 1300 according to an embodiment. Dilation tool 1300 includes a dilation head 1310 and an actuation portion 1305 (FIGS. 6 and 7) including an outer shaft 1360, a drive shaft 1370 (see FIGS. 12 and 13), a lock tab 1380, a handle 1386 and an indicator 1390. FIG. 6 illustrates the dilation tool 1300 with the dilation head 1310 in a first configuration (e.g., unexpanded or collapsed) and with the lock tab 1380 secured to the outer shaft 1360, preventing the handle 1386 from moving relative to the outer shaft 1360. FIG. 7 illustrates the dilation tool 1300 with the dilation head 1310 in a second configuration (e.g. expanded) with the lock tab 1380 removed and the indicator 1390 slid partially outside of the handle 1386.

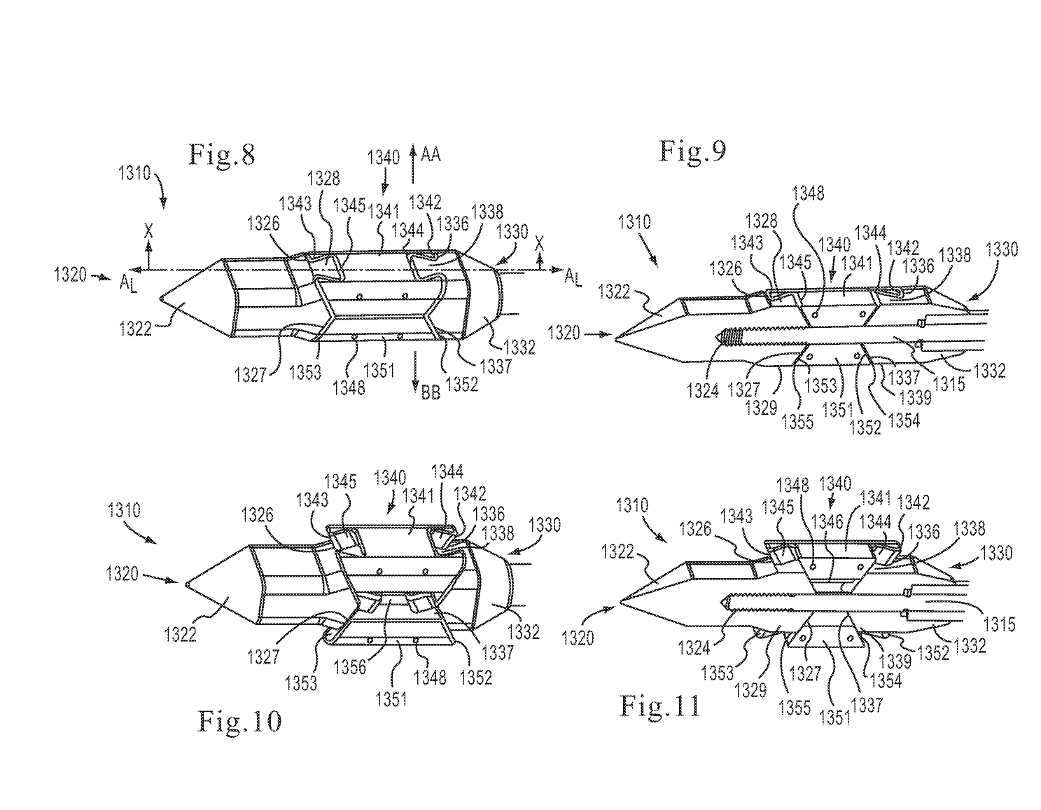

The dilation head 1310 of dilation tool 1300 has a distal end portion 1320, a proximal end portion 1330 and a central portion 1340. Various components of dilation head 1310 are matingly and movably coupled together, for example, by mating protrusions and grooves of the type shown and described in U.S. Patent Application Attorney Docket No. KYPH-040/03US, which is incorporated herein by reference in its entirety. The central portion 1340 is coupled between the distal end portion 1320 and the proximal end portion 1330. The dilation head 1310 also defines a lumen 1315 (see FIG. 9) that is defined collectively by the proximal end portion 1330, the central portion 1340 and the distal end portion 1320. The lumen 1315 is configured to allow a proximal end portion 3172 of the drive shaft 3170 to pass through the first dilation head 1310 when the dilation head 1310 is in the first configuration.

As shown in FIGS. 8-11, the distal end portion 1320 of dilation head 1310 includes a tapered surface 1322, a first engagement surface 1326, a second engagement surface 1327, a first protrusion 1328 and a second protrusion 1329. The distal end portion 1320 of dilation head 1310 also defines a threaded portion 1324 (see FIG. 9) that is configured to threadedly engage a threaded portion 1378 of a distal end portion 1376 of the drive shaft 1370 as described below. The threaded portion 1324 has a predetermined length such that the longitudinal travel of the drive shaft 1370 within the threaded portion is limited. Similarly stated, the threaded portion 1324 is a “blind hole” to limit the longitudinal distance that the drive shaft 1370 can move relative to the distal end portion 1320 of the dilation head 1310. In this manner, the amount of distraction and/or measurement by the tool 1300 can be limited.

The first engagement surface 1326 of the distal end portion 1320 is angularly offset from a longitudinal axis A_{L} defined by the dilation head 1310 by an angle between 0 degrees and 90 degrees. Similarly, the second engagement surface 1327 of the distal end portion 1320 is angularly offset from the longitudinal axis ΔL by an angle between 0 degrees and 90 degrees. Although the angle of the first engagement surface 1326 is shown as being equal, but in an opposite direction to the angle of the second engagement surface 1327 (e.g., the angle of the first engagement surface is +110 degrees and the angle of the second engagement surface 1327 is −110 degrees), in other embodiments, the angle of the first engagement surface 1326 and the angle of the second engagement surface 1327 can be different. As described in more detail herein, the angular offset of the first engagement surface 1326 and the angular offset of the second engagement surface 1327 are associated with moving the dilation head 1310 between a first configuration (FIGS. 6, 8 and 9) and a second configuration (FIGS. 7, 10 and 11).

The first protrusion 1328 of the distal end portion 1320 has an undercut such that the first dilation member 1341 of the central portion 1340 of the dilation head 1310 can be slidably coupled to the distal end portion 1320 of the dilation head 1310. Similarly, the second protrusion 1329 of the distal end portion 1320 has an undercut such that the second dilation member 1351 of the central portion 1340 can be slidably coupled to the distal end portion 1320. More particularly, the first protrusion 1328 and second protrusion 1329 each have a trapezoidal cross-sectional shape. In some embodiments, for example, the first protrusion 1328 and second protrusion 1329 can each have a dovetail protrusion.

The proximal end portion 1330 of dilation head 1310 includes a tool engagement member 1332, a first engagement surface 1336, a second engagement surface 1337, a first protrusion 1338 and a second protrusion 1339. The first engagement surface 1336 of the proximal end portion 1330 is angularly offset from the longitudinal axis A_{L} of the dilation head 1310 by an angle between 0 degrees and 90 degrees. Similarly, the second engagement surface 1337 of the proximal end portion 1330 is angularly offset from the longitudinal axis A_{L} by an angle between 0 degrees and 90 degrees. Although the angle of the first engagement surface 1336 is shown as being equal, but in an opposite direction to the angle of the second engagement surface 1337 (e.g., the angle of the first engagement surface 1336 is +110 degrees and the angle of the second engagement surface 1337 is −110 degrees), in other embodiments, the angle of the first engagement surface 1336 and the angle of the second engagement surface 1337 can be different. As described in more detail herein, the angular offset of the first engagement surface 1336 and the angular offset of the second engagement surface 1337 are associated with moving the dilation head 1310 between a first configuration (FIGS. 6, 8 and 9) and a second configuration (FIGS. 7, 10 and 11).

The first protrusion 1338 of the proximal end portion 1330 has an undercut such that the first dilation member 1341 of the central portion 1340 of the dilation head 1310 can be slidably coupled to the proximal end portion 1330 of the dilation head 1310. Similarly, the second protrusion 1339 of the proximal end portion 1330 has an undercut such that the second dilation member 1351 of the central portion 1340 can be slidably coupled to the proximal end portion 1330. More particularly, the first protrusion 1338 and second protrusion 1339 each have a trapezoidal cross-sectional shape. In some embodiments, the first protrusion 1338 and second protrusion 1339 can each have a dovetail protrusion.

The central portion 1340 of dilation head 1310 includes a first dilation member 1341 and a second dilation member 1351. The first dilation member 1341 includes a proximal engagement surface 1342 and a distal engagement surface 1343. The central portion 1340 of the dilation head 1310 can also include radiotranslucent holes 1348 that are viewable on an imaging device (e.g., a fluoroscope). The radiotranslucent holes 1348 can be used as markers to help position the dilation head 1310 with relative to the spinous processes. The first dilation member 1341 defines a notch 1346 (see FIG. 11) configured to allow the drive shaft 1370 to pass through the first dilation member 1341.

The distal engagement surface 1343 of the first dilation member 1341 defines a plane that is angularly offset from the longitudinal axis A_{L} of the dilation head 1310 by an angle between 90 degrees and 180 degrees. Moreover, the angular offset of the distal engagement surface 1343 of the first dilation member 1341 is supplementary with the angular offset of the first engagement surface 1326 of the distal end portion 1320 (i.e., the angles sum to 180 degrees). Similarly stated, the distal engagement surface 1343 is substantially parallel to the first engagement surface 1326 of the distal end portion 1320. Accordingly, the first dilation member 1341 is slidably disposed against the distal end portion 1320.

The distal engagement surface 1343 of the first dilation member 1341 defines a distal groove 1345 having a trapezoidal cross-sectional shape. In this embodiment, the distal groove 1345 has a dovetail shape that corresponds to the shape of the first protrusion 1328 of the distal end portion 1320. The distal groove 1345 is configured to receive and to slide along the first protrusion 1328 of the distal end portion 1320. The undercut of the first protrusion 1328 of the distal end portion 1320 slidably maintains the first protrusion 1328 of the distal end portion 1320 within the distal groove 1345. The distal groove 1345 of the distal engagement surface 1343 and the protrusion 1328 of the distal end portion 1320 collectively allow movement of the first dilation member 1341, with respect to the distal end portion 1320, in a direction substantially parallel to the proximal engagement surface 1342 of the first dilation member 1341. Moreover, the distal groove 1345 of the distal engagement surface 1343 and the protrusion 1328 of the distal end portion 1320 collectively limit movement of the first dilation member 1341 with respect to the distal end portion 1320, in a direction substantially normal to the proximal engagement surface 1342 of the first dilation member 1341. The distal engagement surface 1343 of the first dilation member 1341 contacts and is configured to slide along the first engagement surface 1326 of the distal end portion 1320 when the distal groove 1345 slides along the first protrusion 1328 of the distal end portion 1320.

The proximal engagement surface 1342 of the first dilation member 1341 defines a plane that is angularly offset from the longitudinal axis A_{L} of the dilation head 1310 by an angle greater than 90 degrees. Moreover, the angular offset of the proximal engagement surface 1342 of the first dilation member 1341 is supplementary with the angular offset of the first engagement surface 1336 of the proximal end portion 1330. For example, the proximal engagement surface 1342 is substantially parallel to the proximal engagement surface 1342 of the proximal end portion 1330. Accordingly, the first dilation member 1341 is slidably disposed against the proximal end portion 1330.

The proximal engagement surface 1342 of the first dilation member 1341 defines a proximal groove 1344 having a trapezoidal cross-sectional shape. In this embodiment, the proximal groove 1344 has a dovetail shape that corresponds to the shape of the first protrusion 1338 of the proximal end portion 1330. The proximal groove 1344 is configured to receive and to slide along the first protrusion 1338 of the proximal end portion 1330. The undercut of the first protrusion 1338 of the proximal end portion 1330 slidably maintains the first protrusion 1336 of the proximal end portion 1330 within the proximal groove 1344. The proximal groove 1344 of the proximal engagement surface 1342 and the protrusion 1338 of the proximal end portion 1330 collectively allow movement of the first dilation member 1341, with respect to the proximal end portion 1330, in a direction substantially parallel to the distal engagement surface 1343 of the first dilation member 1341. Moreover, the proximal groove 1344 of the proximal engagement surface 1344 and the protrusion 1338 of the proximal end portion 1330 collectively limit movement of the first dilation member 1341 with respect to the proximal end portion 1330, in a direction substantially normal to the distal engagement surface 1343 of the first dilation member 1341. The proximal engagement surface 1342 of the first dilation member 1341 contacts and is configured to slide along the first engagement surface 1336 of the proximal end portion 1330 when the proximal groove 1344 slides along the first protrusion 1336 of the proximal end portion 1330.

Likewise, the second dilation member 1351 of the central portion 1340 includes a proximal engagement surface 1352 and a distal engagement surface 1353. The second dilation member 1351 defines a notch 1356 (see FIG. 10) configured to allow the drive shaft 1370 to pass through the first dilation member 1341. The proximal engagement surface 1352 defines a proximal groove 1354 and the distal engagement surface 1353 defines a distal groove 1355. The second dilation member 1351 is configured similar to the first dilation member 1341 and is therefore not described in detail herein.

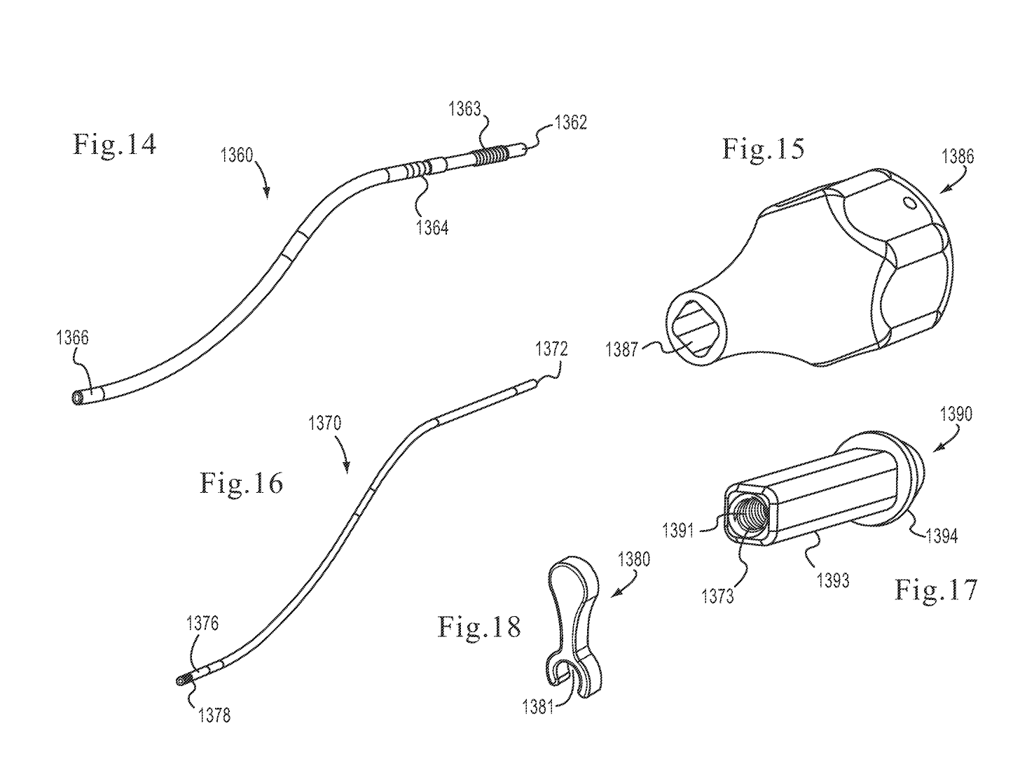

FIGS. 12 and 13 are each a cross-sectional view of the dilation tool 1300 (with the dilation head 1310 in the first configuration) to illustrate the connection between the dilation head 1310 and the actuation portion of the dilation tool 1310. The various components of the actuation portion of the dilation tool 1300 are shown individually in FIGS. 14-18. The outer shaft 1360 of the dilation tool 1300 is shown in FIG. 14. The outer shaft 1360 includes a proximal end portion 1362 and a distal end portion 1366. The proximal end portion 1362 of the outer shaft 1360 includes a threaded portion 1363 configured to be coupled to a threaded portion 1373 of the indicator 1390 described in more detail below. At least a portion of the outer shaft 1360 can be formed with a flexible material such that it can bend and/or assume a curved shape. In other embodiments, however, the outer shaft 1360 can be substantially rigid, and can be formed to include a curved shape as desired. In some embodiments, the outer shaft 1360 can be formed at least in part with a flexible coil. Multiple markers 1364 are disposed on an outer surface of the outer shaft 1360 (see e.g., FIGS. 6 and 14). Distal end portion 1366 of the outer shaft 1360 is configured to be coupled to the tool engagement member 1332 of the distal end portion 1320 of the dilator head 1310. The outer shaft 1360 of the dilation tool 1300 defines a lumen 1361 (see FIG. 13) configured to allow the drive shaft 1370 of the dilation tool to be disposed within.

The drive shaft 1370 of the dilation tool 1300 is shown in FIG. 16. The drive shaft 1370 of the dilation tool 1300 includes a proximal end portion 1372 and a distal end portion 1376. The drive shaft 1370 of the dilation tool 1300 is configured to be disposed within the lumen 1361 defined by the outer shaft 1360 of the dilation tool 1300. The inner shaft 1370 can be formed at least in part with a flexible material. For example, at least a portion of the inner shaft 1370 can be formed with a coil. This allows the inner shaft 1370 to be actuatable while disposed within the outer shaft 1360, for example, when the outer shaft 1360 is curved. The proximal end portion 1372 is disposed within a lumen 1387 defined by the handle 1386 (see FIG. 15) of the dilation tool 1300 and is coupled to the handle 1386 of the dilation tool 1300. A retaining member 1377 is disposed at the distal end portion 1376 of the drive shaft 1370 (see FIG. 13) and a retaining member 1375 is disposed at the proximal end portion 1372 of the drive shaft 1370 to prevent axial movement of the drive shaft 1370 relative to the outer shaft 1360. The retaining members 1377 and 1375 can be any suitable structure configured to limit the axial movement of the drive shaft 1370 relative to the outer shaft 1360, such as, for example, a snap ring, an E-ring, C-clip, a set screw, a detent configured to be retained within a recess, and/or the like. A threaded portion 1378 of the distal end portion 1376 of the drive shaft 1370 is configured to engage the threaded portion 1324 of the distal end portion 1320 of the dilation head 1310.

The lock tab 1380 of the dilation tool 1300 is shown in FIG. 18. The lock tab 1380 of the dilation tool 1300 defines a notch 1381 configured to engage a cut-out portion 1383 of the outer shaft 1360 of the dilation tool 1300 as shown in FIGS. 6, 12 and 13. When engaged with the outer shaft 1360, the lock tab 1380 is disposed against the indicator 1390 of the dilation tool 1300, which prevents the indicator 1390 and the handle 1386 from rotating with respect to the outer shaft 1360.

The handle 1386 of the dilation tool 1300 is shown in FIG. 15. The handle 1386 defines a lumen 1387 configured to receive an elongate portion 1393 (see FIG. 17) of the indicator 1390 of the dilation tool 1300 as shown in FIGS. 12, 13 and 17. The elongate portion 1393 is keyed into the lumen 1387 such that the handle 1386 and the indicator 1390 do not rotate relative to each other, but the indicator 1390 can move axially relative to the handle 1386. The handle 1386 is configured to rotate the drive shaft 1370 relative to the outer shaft 1360 to move the dilation head 1310 between the first configuration and the second configuration. In some embodiment, the handle 1386 can rotate about a portion of a centerline of the outer shaft 1360. For example, if the outer shaft 1360 is non-linear or curved, the outer shaft 1360 will have a non-linear centerline and the handle 1386 can rotate about a portion of the outer shaft 1360 that has a substantially linear centerline.

The indicator 1390 of the dilation tool 1300 is shown in FIG. 17. The indicator 1390 of the dilation tool 1300 defines a lumen 1391 that extends through the elongate portion 1393 and through a distal end portion 1394 of the indicator 1390. The proximal end portion 1362 of the outer shaft 1360 is received through an opening 1395 (see FIG. 13) defined by the distal end portion 1394 of the indicator 1390 and the threaded portion 1363 of the outer shaft 1360 matingly engages the a threaded portion 1373 defined within the lumen 1391 of the indicator 1390.

The indicator 1390 is used to provide an indication to the user of the amount or size of dilation or distraction that has been produced by the tool 1300. As the handle 1386 of the dilation tool 1300 is rotated, the indicator 1390 will rotate relative to the outer shaft 1360 and is drawn longitudinally along the threaded portion 1363 of the outer shaft 1360. The distance that the indicator 1390 has moved longitudinally can correspond to the amount of distraction produced and/or the size of the cavity being measured. For example, when used to distract adjacent spinous processes, a location of the indicator 1390 relative to the markers 1364 on the outer shaft 1360 can indicate the distance the indicator 1390 has moved and the corresponding distance between and/or amount of distraction of the adjacent spinous processes. Similarly, when used to measure the space between adjacent spinous processes and/or between vertebral end plates, a location of the indicator 1390 relative to the markers 1364 on the outer shaft 1360 can indicate the distance the indicator 1390 has moved and the corresponding distance between the adjacent spinous processes and/or the vertebral end plates. In some embodiments, the markers 1364 can include numerical measurements of the amount of distraction and/or size of the space being measured. In other embodiments, the markers 1364 can correspond to different spacers that can be disposed within the space based on the amount of distraction and/or size of the space being measured Similarly stated, in some embodiments, the markers 1364 can include qualitative indications (e.g., part numbers, spacer designations or the like) associated with the amount of distraction and/or size of the space being measured.

The threaded portion 1373 of the indicator 1390 can have the same pitch as the threaded portion 1378 of the distal end portion 1376 of the drive shaft 1370 such that the distance the distal end portion 1376 travels within the distal head 1310 correlates to the distance the indicator 1390 travels along the outer shaft 1360. In some embodiments, the pitch of the threaded portion 1373 is different than the pitch of the threaded portion 1378 to change the correlation to the indicator 1390.

In use, with the dilation head 1310 in the first configuration and the lock tab 1380 engaged with the outer shaft 1360 (see e.g., FIG. 6), the dilation tool 1300 is inserted percutaneously to a location within a patient's body. For example, the dilation tool 1300 can be disposed within a space between a pair of adjacent spinous processes. The distal end portion 1320 of the dilation head 1310 is inserted first and is moved until the central portion 1340 of the dilation head 1310 is positioned in the space between the adjacent spinous processes.

Once between the spinous processes, the dilation tool 1300 can be moved from the first configuration to the second configuration (see e.g., FIG. 7). This is accomplished by removing the lock tab 1380 from the outer shaft 1360 and rotating the handle 1386. Rotation of the handle 1386 causes the drive shaft 1370 to rotate, which in turn causes the distal end portion 1320 of the dilation head 1310 to move toward the proximal end portion 1330 of the dilation head 1310. The distal end portion 1320 of the dilation head 1310, and the proximal end portion 1330 of the dilation head 1310 exert a force on the first dilation member 1341 of the central portion 1340 of the dilation head 1310 and on the second dilation member 1351 of the central portion 1340 of the dilation head 1310.

The force causes the first dilation member 1341 of the central portion 1340 of the dilation head 1310 to move in the direction AA as shown in FIG. 8 with respect to the distal end portion 1320 of the dilation head 1310 and the proximal end portion 1330 of the dilation head 1310. Likewise, the force causes the second dilation member 1351 of the central portion 1340 of the dilation head 1310 to move in the direction BB as shown in FIG. 8 with respect to the distal end portion 1320 of the dilation head 1310 and the proximal end portion 1330 of the dilation head 1310. The force exerted by the first dilation member 1341 and the second dilation member 1351 on the adjacent spinous processes, causes the spinous processes to distract.

As the handle 1386 of the dilation tool 1300 is rotated, the indicator 1390 of the dilation tool 1300 rotates and moves longitudinally with respect to the outer shaft 1360 of the dilation tool 1300 as described above. The movement of the indicator 1390 corresponds to a distance between the adjacent spinous processes, at least a portion of which also corresponds to the amount of distraction produced between the adjacent spinous processes. When a desired amount of distraction has been achieved, the dilation tool 1300 can be moved back to the first configuration and removed from the patient's body. To do this, the handle 1386 of the dilation tool 1300 can be rotated in an opposite direction causing the dilation tool 1300 to return to the first configuration.

In some embodiments, the handle 1386 of the dilation tool 1300 can include a torque limiting mechanism (not shown) to prevent over-distraction of a particular space. For example, in some embodiments the dilation tool 1300 can be used to create a void within a disc space and/or repair a bone fracture. A torque limiting mechanism can allow the user to apply a force to the bone structure up to a predetermined maximum value. In this manner, the dilation tool 1300 can prevent over-distraction during use.

Although the dilation tool 1300 is shown is being movable between a first configuration (FIG. 8) and a second configuration (FIG. 10), the dilation tool 1300 can be maintained in any number of different configurations. For example, the dilation tool 1300 can be maintained in any suitable configuration between the first configuration and the second configuration. Said another way, the dilation tool 1300 can be placed in an infinite number of different configurations between the first configuration and the second configuration. Thus, the space between the spinous processes can be distracted by the first dilation member 1341 and the second dilation member 1351 by any desired amount within a predetermined range. In this manner, a single dilation tool 1300 can be used within a wide range locations within the body requiring different amounts of distraction and/or measurement.

Moreover, this arrangement allows the amount of distraction and/or measurement to be varied in situ over time. For example, in some embodiments, the amount of distraction and/or measurement can be varied within a range of approximately 8 mm to 16 mm. Within this range, the size of the central portion 1340 can be adjusted to any desired amount by rotating the handle 1386 a predetermined amount, as described above. In other embodiments, the range of distraction and/or measurement can be approximately 4 mm (e.g., a range from 5 mm to 9 mm, a range from 12 mm to 16 mm, or the like). In yet other embodiments, the range of distraction and/or measurement can be approximately 3 mm (e.g., a range from 10 mm to 13 mm, a range from 12 mm to 15 mm, or the like).

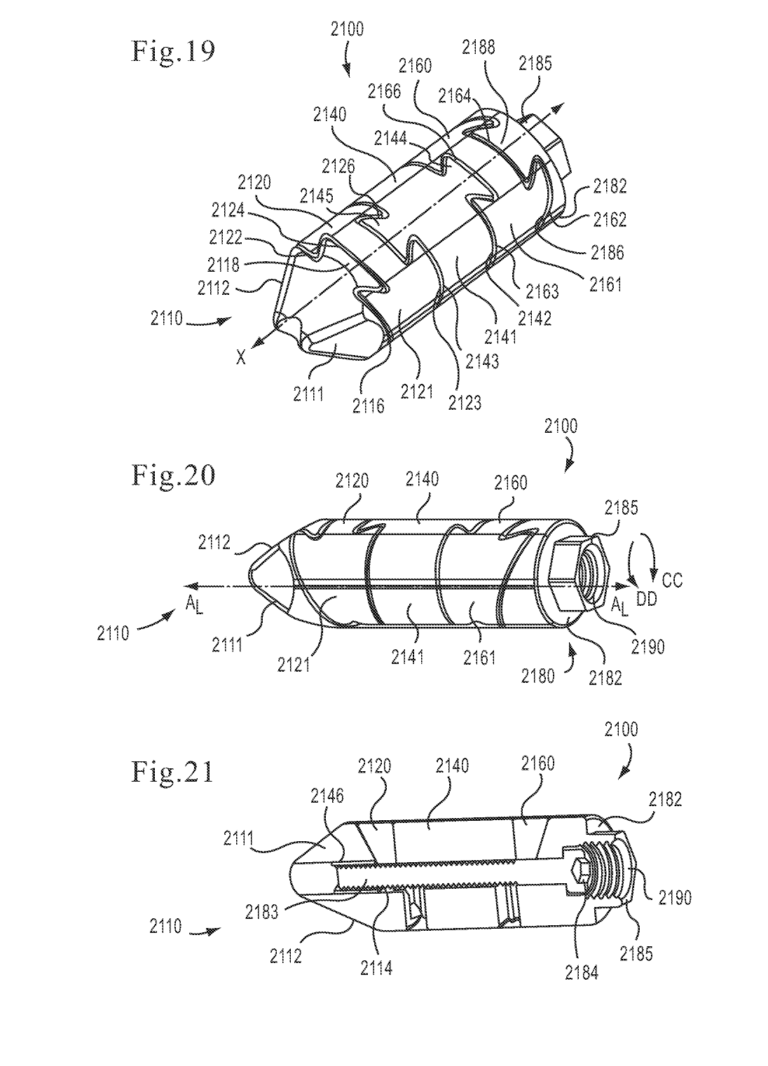

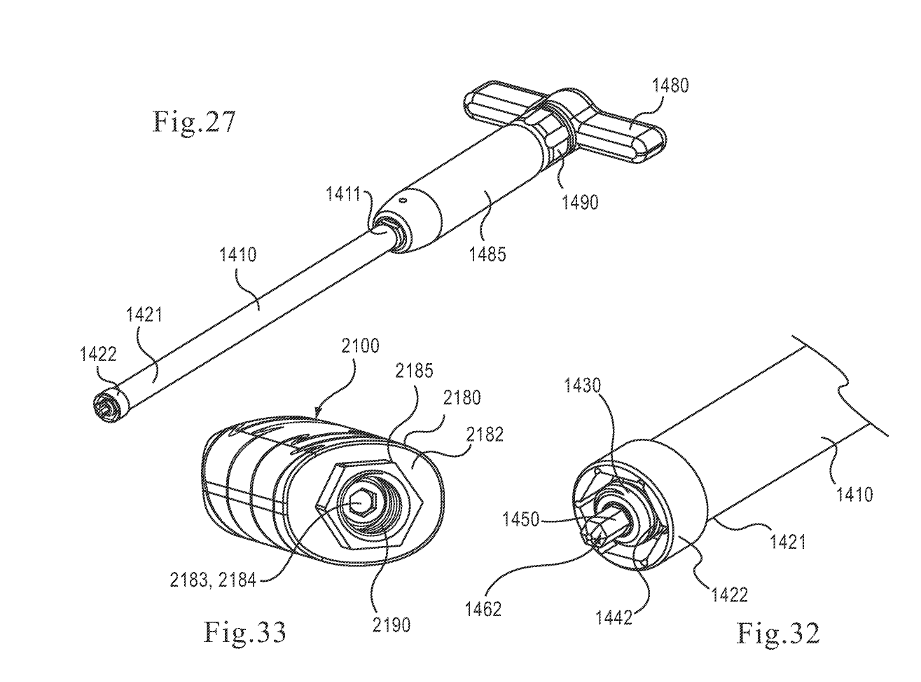

FIGS. 27-41 illustrate an implant insertion/removal tool 1400, according to another embodiment of the invention. To better illustrate the function and use of the implant insertion/removal tool 1400, an example implant is described with reference to FIGS. 19-26.

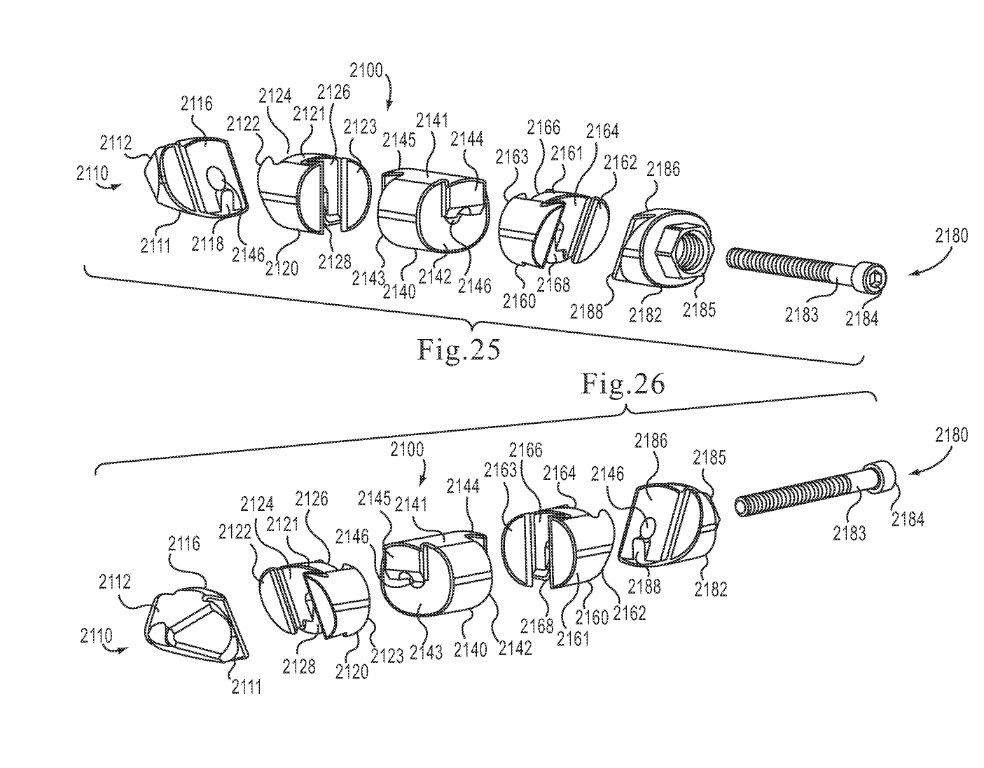

FIGS. 19-26 illustrate an implant 2100, according to an embodiment. Implant 2100 includes a distal end portion 2110, a central portion 2140 and a proximal end portion 2180. At least a portion of the central portion 2140 is disposed in a space between the distal end portion 2110 and the proximal end portion 2180. The implant 2100 defines a lumen 2146 (see e.g., FIGS. 25 and 26) and includes a drive screw 2183 disposed within the lumen 2146. Drive screw 2183 has a tool head 2184 configured to mate with and/or receive a tool for rotating the drive screw 2183, as further described below.

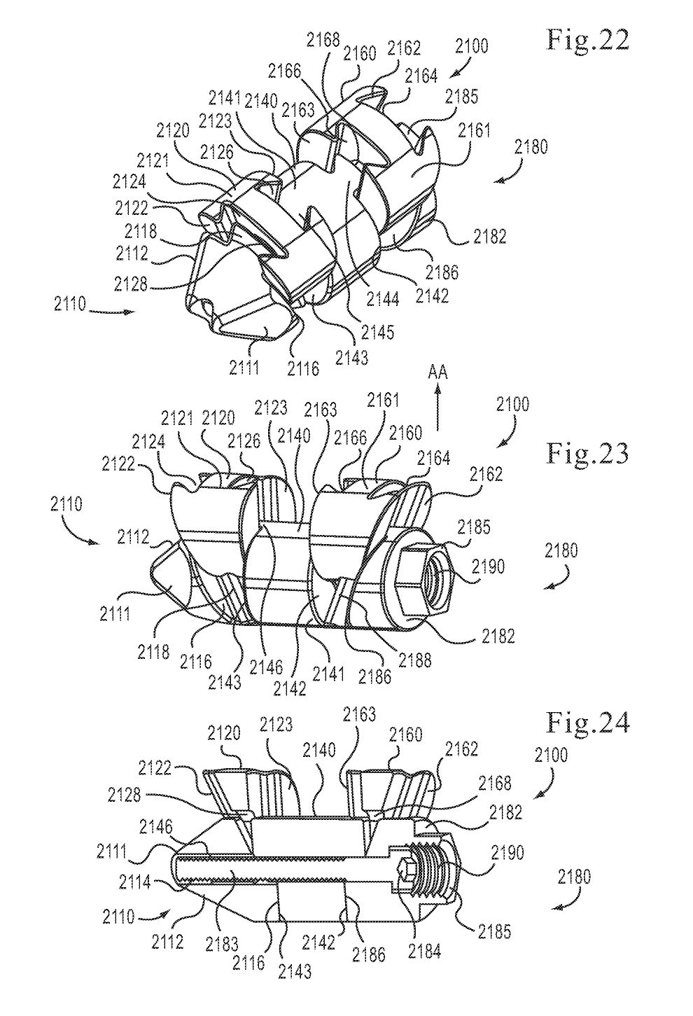

The distal end portion 2110 of implant 2100 includes an actuator 2111 and a distal retention member 2120. Actuator 2111 includes a tapered surface 2112, a threaded portion 2114 (see FIG. 21), and an engagement surface 2116. The threaded portion 2114 is disposed fixedly within the lumen 2146 and is configured to receive the drive screw 2183, as described above. The engagement surface 2116 of the actuator 2111 is angularly offset from the longitudinal axis A_{L} of the implant 2100 by an angle between 0 degrees and 90 degrees. As described in more detail herein, the angular offset of the engagement surface 2116 is associated with moving the implant 2100 between a first configuration (FIG. 19) and a second configuration (FIG. 22). The engagement surface 2116 includes a protrusion 2118 having an undercut such that the distal retention member 2120 can be coupled to the actuator 2111. More particularly, the protrusion 2118 has a trapezoidal cross-sectional shape. In some embodiments, the protrusion 2118 is a dovetail protrusion.

Distal retention member 2120 includes an outer surface 2121, a first engagement surface 2122, and a second engagement surface 2123 opposite the first engagement surface 2122. The distal retention member 2120 defines a notch 2128 (see FIG. 24) configured to allow the drive screw 2183 to pass through the distal retention member 2120 when the implant 2100 is in the first configuration. The first engagement surface 2122 of the distal retention member 2120 defines a plane that is angularly offset from the longitudinal axis A_{L} of the implant 2100 by an angle between 90 degrees and 180 degrees. Moreover, the first engagement surface 2122 of the distal retention member 2120 is substantially parallel to the engagement surface 2116 of the actuator 2111. Accordingly, the distal retention member 2120 is slidably disposed against actuator 2111.

The first engagement surface 2122 of the distal retention member 2120 defines a first groove 2124 having a trapezoidal cross-sectional shape. In this embodiment, the first groove 2124 has a dovetail shape that corresponds to the shape of the protrusion 2118 of the actuator 2111. The first groove 2124 of the first engagement surface 2122 and the protrusion 2118 of the actuator 2111 collectively allow movement of the distal retention member 2120, with respect to the actuator 2111, in a direction substantially parallel to the second engagement surface 2123 of the distal retention member 2120. Moreover, the first groove 2124 of the first engagement surface 2122 and the protrusion 2118 of the actuator 2111 collectively limit movement of the distal retention member 2120, with respect to the actuator 2111, in a direction substantially normal to the second engagement surface 2123 of the distal retention member 2120. The first engagement surface 2122 of the distal retention member 2120 contacts and is configured to slide along the engagement surface 2116 of the actuator 2111 when the first groove 2124 slides along the protrusion 2118 of the actuator 2111.

The second engagement surface 2123 of the distal retention member 2120 is substantially parallel to the distal engagement surface 2143 of the central portion 2140 and defines a plane substantially normal to the longitudinal axis A_{L} of the implant 2100. The second engagement surface 2123 of the distal retention member 2120 defines a second groove 2126 having a trapezoidal cross-sectional shape. In this embodiment, the second groove 2126 has a dovetail shape that corresponds to the shape of the distal protrusion 2145 of the central portion 2140. The second groove 2126 of the second engagement surface 2123 and the distal protrusion 2145 of the central body 2140 collectively limit movement of the distal retention member 2120, with respect to the central portion 2140, in a direction substantially normal to the second engagement surface 2123 of the distal retention member 2120. The second engagement surface 2123 of the distal retention member 2120 is slidably disposed against and/or coupled to the central portion 2140 of the implant 2100, as described in more detail herein.

Proximal end portion 2180 of implant 2100 includes a tool engagement member 2182 and a proximal retention member 2160. Tool engagement member 2182 is configured to mate with and/or receive an insertion tool. Tool engagement member 2182 includes an engagement surface 2186 and a hex portion 2185. The hex portion 2185 includes a hexagonal shaped outer surface configured to be matingly received within a portion of an insertion tool. In this manner, the hex portion 2185 of the tool engagement member 2182 can limit rotational motion of the implant 2100 about the longitudinal axis A_{L}, when the implant 2100 is coupled to an insertion tool. In some embodiments, the hexagonal shaped outer surface of the hex portion 2185 can be configured to facilitate the docking of the insertion tool (not shown) onto the hex portion 2185 of the implant 2100. For example, in some embodiments, the outer surface of the hex portion 2185 can include a lead-in chamfer, a tapered portion and/or a beveled edge to facilitate the docking of the insertion tool onto the hex portion 2185 of the implant 2100.

The hex portion 2185 defines a threaded portion 2190. The threaded portion 2190 is configured to mate with and/or receive a corresponding threaded portion of an insertion tool. In this manner, the threaded portion 2190 can limit axial movement of the implant 2100, with respect to the insertion tool, when the implant 2100 is inserted into a body of a patient, as described in further detail below. Moreover, when the shaft 1430 of the insertion tool is coupled within the threaded portion 2190, movement of the drive screw 2183 along the longitudinal axis relative to the tool engagement member 2182 is limited. In this manner, the coupling of an insertion tool 1400 within the threaded portion 2190 can prevent the drive screw 2183 from moving, thereby maintaining the implant 2100 in the first configuration. In other embodiments, the threaded portion 2190 can include a retainer (e.g., a snap ring, an E-ring or the like) to prevent translation of the drive screw 2183 relative to the tool engagement member 2182.

The engagement surface 2186 of the tool engagement member 2182 is angularly offset from the longitudinal axis A_{L} of the implant 2100 by an angle between 0 degrees and 90 degrees. The engagement surface 2186 includes a protrusion 2188 having an undercut such that the proximal retention member 2160 can be coupled to the tool engagement member 2182. More particularly, the protrusion 2188 has a trapezoidal cross-sectional shape. In this embodiment, the protrusion 2188 is a dovetail protrusion.

Proximal retention member 2160 includes an outer surface 2161, a first engagement surface 2162, and a second engagement surface 2163 opposite the first engagement surface 2162. The proximal retention member 2160 defines a notch 2168 (see FIG. 26) configured to allow the drive screw 2183 to pass through the proximal retention member 2160 when the implant 2100 is in the first configuration. The first engagement surface 2162 of the proximal retention member 2160 defines a plane that is angularly offset from the longitudinal axis A_{L} of the implant 2160 by an angle between 90 degrees and 180 degrees. Moreover, the first engagement surface 2162 of the proximal retention member 2160 is substantially parallel to the engagement surface 2186 of the tool engagement member 2182. Accordingly, the proximal retention member 2160 is slidably disposed against the tool engagement member 2182.

The first engagement surface 2162 of the proximal retention member 2160 defines a first groove 2164 having a trapezoidal cross-sectional shape. In this embodiment, the first groove 2164 has a dovetail shape that corresponds to the shape of the protrusion 2188 of the tool engagement member 2182. The undercut of the protrusion 2188 of the tool engagement member 2182 slidably maintains the protrusion 2188 of the tool engagement member 2182 within the first groove 2164. More particularly, the first groove 2164 of the first engagement surface 2162 and the protrusion 2188 of the tool engagement member 2182 collectively allow movement of the proximal retention member 2160, with respect to the tool engagement member 2182, in a direction substantially parallel to the second engagement surface 2163 of the proximal retention member 2160. Moreover, the first groove 2164 of the first engagement surface 2162 and the protrusion 2188 of the tool engagement member 2182 collectively limit movement of the proximal retention member 2160, with respect to the tool engagement member 2182, in a direction substantially normal to the second engagement surface 2163 of the proximal retention member 2160. The first engagement surface 2162 of the proximal retention member 2160 contacts and is configured to slide along the engagement surface 2186 of the tool engagement member 2182 when the first groove 2164 of the proximal retention member 2160 slides along the protrusion 2188 of the tool engagement member 2182.

The second engagement surface 2163 of the proximal retention member 2160 is substantially parallel to the proximal engagement surface 2142 of the central portion 2140 and defines a plane substantially normal to the longitudinal axis A_{L} of the implant 2100. The second engagement surface 2163 of the proximal retention member 2160 defines a second groove 2166 having a trapezoidal cross-sectional shape. In this embodiment, the second groove 2166 has a dovetail shape that corresponds to the shape of the proximal protrusion 2144 of the central portion 2140. The second groove 2166 of the second engagement surface 2163 and the proximal protrusion 2144 of the central portion 2140 collectively limit movement of the proximal retention member 2160, with respect to the central body 2140, in a direction substantially normal to the second engagement surface 2163 of the proximal retention member 2160. The second engagement surface 2163 of the proximal retention member 2160 is slidably disposed against and/or coupled to the central portion 2140 of the implant 2100, as described in more detail herein.

The central portion 2140 of implant 2100 includes a proximal engagement surface 2142, a distal engagement surface 2143, a proximal protrusion 2144, a distal protrusion 2145 and an outer surface 2141. The distal retention member 2120 is slidably coupled to the central portion 2140. The second groove 2126 of the distal retention member 2120 is configured to slidingly receive the distal protrusion 2145 of the central portion 2140. The distal protrusion 2145 of the central portion 2140 has a dovetail shape slidably maintaining it within the second groove 2126 of the distal retention member 2120. The second engagement surface 2123 of the distal retention member 2120 contacts and is configured to slide along the distal engagement surface 2143 of the central portion 2140 when the second groove 2126 of the distal retention member 2120 slides along the distal protrusion 2145 of the central portion 2140.

Similarly, the proximal retention member 2160 is slidably coupled to the central portion 2140. The second groove 2166 of the proximal retention member 2160 is configured to slidingly receive the proximal protrusion 2144 of the central portion 2140. The proximal protrusion 2144 of the central portion 2140 has a dovetail shape slidably maintaining it within the second groove 2166 of the proximal retention member 2160. The second engagement surface 2163 of the proximal retention member 2160 contacts and is configured to slide along the proximal engagement surface 2142 of the central portion 2140 when the second groove 2166 of the proximal retention member 2160 slides along the proximal protrusion 2144 of the central portion 2140.

The implant 2100 has a first configuration (FIG. 19) and a second configuration (FIG. 23). When the implant 2100 is in the first configuration, the proximal end portion 2180, the distal end portion 2110 and the central portion 2140 are substantially coaxial (i.e., substantially share a common longitudinal axis). As described above, the implant 2100 can be moved between the first configuration and the second configuration by rotating the drive screw 2183. When the drive screw 2183 is rotated as indicated by the arrow CC in FIG. 20, the drive screw 2183 moves the actuator 2111 and the tool engagement member 2182 toward the central portion 2140. The engagement surface 2116 of the actuator 2111 exerts an axial force on the first engagement surface 2122 of the distal retention member 2120. Because the engagement surface 2116 of the actuator 2111 is at an acute angle with respect to the longitudinal axis A_{L}, a component of the axial force transmitted via the engagement surface 2116 to the first engagement surface 2122 of the distal retention member 2120 has a direction as shown by the arrow AA in FIG. 23. Said another way, a component of the force exerted by the actuator 2111 on the distal retention member 2120 has a direction that is substantially normal to the longitudinal axis A_{L}. This force causes the distal retention member 2120 to slide on the engagement surface 2116 of the actuator 2111 causing the distal retention member 2120 to move in the direction AA and into the second configuration. Once the distal retention member 2120 slides on the engagement surface 2116 of the actuator 2111 a predetermined distance, a portion of the engagement surface 2116 of the actuator 2111 contacts a portion of the distal engagement surface 2143 of the central portion 2140 preventing the distal retention member 2120 from sliding further.

Similarly, when the drive screw 2183 is rotated as indicated by the arrow CC in FIG. 20, the engagement surface 2186 of the tool engagement member 2182 exerts an axial force on the first engagement surface 2162 of the proximal retention member 2160. Because the engagement surface 2186 of the tool engagement member 2182 is at an acute angle with respect to the longitudinal axis A_{L}, a component of the axial force transmitted via the engagement surface 2186 to the first engagement surface 2162 of the proximal retention member 2160 has a direction as shown by the arrow AA in FIG. 23. Said another way, a component of the force exerted by the tool engagement member 2182 on the proximal retention member 2160 has a direction that is substantially normal to the longitudinal axis A_{L}. This force causes the proximal retention member 2160 to slide on the engagement surface 2186 of the tool engagement member 2182 causing the proximal retention member 2160 to move in the direction AA and into the second configuration. Once the proximal retention member 2160 slides on the engagement surface 2186 of the tool engagement member 2180 a predetermined distance, a portion of the engagement surface 2186 of the tool engagement member 2180 contacts the proximal engagement surface 2142 of the central portion 2140 preventing the proximal retention member 2160 from sliding further. When the implant 2100 is in the second configuration the distal retention member 2120 and/or the proximal retention member 2160 are offset from the central portion 2140 in a direction substantially normal to the longitudinal axis A_{L}.

The insertion tools described below can include an actuator configured to be inserted into the tool head 2184 of the drive screw 2183 to rotate the drive screw 2183 about the longitudinal axis A_{L}. This arrangement allows the drive screw 2183 to be rotated without rotating the other portions of the implant 2100. Accordingly, the implant 2100 can be inserted into, repositioned within and/or removed from a body, as described above.

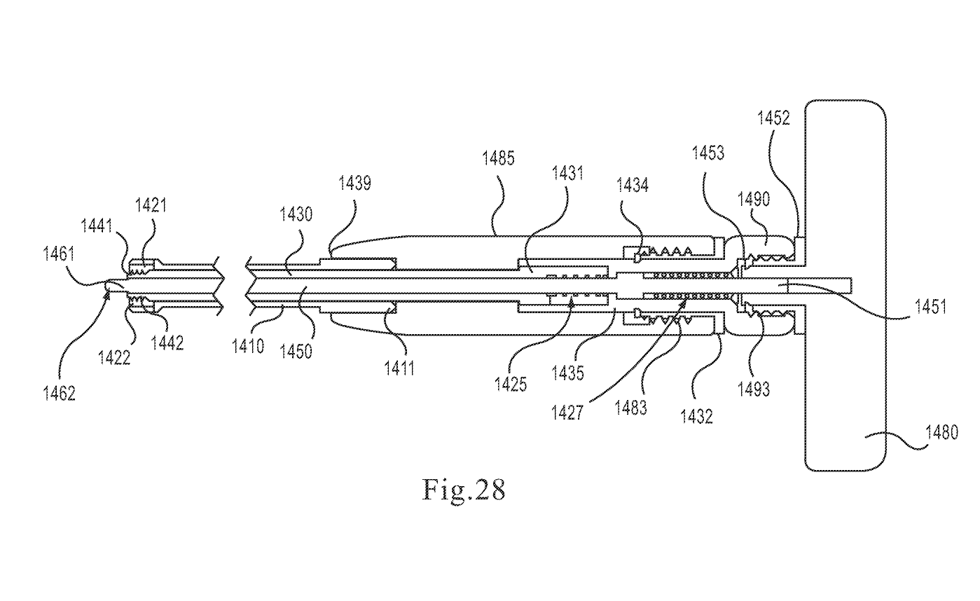

Referring now to FIGS. 27-41, the implant insertion/removal tool 1400 is described in reference to being coupled to the implant 2100 described above. It should be understood that the insertion/removal tool 1400 can be used to insert/remove and/or actuate other types of implants. FIG. 27 is a perspective view of the implant insertion/removal tool 1400 and FIG. 28 is a cross-sectional view of the implant insertion/removal tool 1400 (also referred to herein as “insertion/removal tool”). As shown in FIGS. 27 and 28 the implant insertion/removal tool 1400 includes an outer shaft 1410, an intermediate shaft 1430, an inner shaft 1450, an actuation handle 1480, a housing 1485 and a release knob 1490.

The actuation handle 1480 is coupled to the inner shaft 1450. The housing 1485 is coupled to the outer shaft 1410, and the release knob 1490 is coupled to the intermediate shaft 1430. The actuation handle 1480, the housing 1485 and the release knob 1490 share a common centerline or longitudinal axis. The actuation handle 1480 can rotate about the longitudinal axis to rotate the inner shaft 1450 independent of the release knob 1490 and the intermediate shaft 1430. The release knob 1490 can rotate about the longitudinal axis to rotate the intermediate shaft 1430 independent of the handle 1480 and the inner shaft 1450.

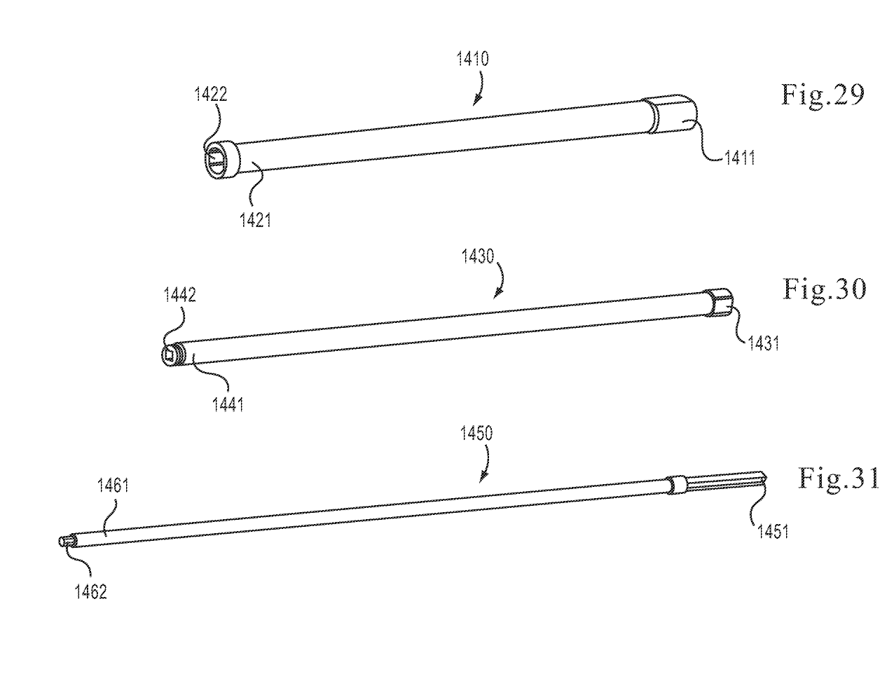

As shown in FIG. 29, the outer shaft 1410 of the implant insertion/removal tool 1400 includes a proximal end portion 1411 and a distal end portion 1421 (see also FIG. 27). Outer shaft 1410 of the implant insertion/removal tool 1400 defines a lumen (not shown) configured to receive intermediate shaft 1430 of the implant insertion/removal tool 1400. As best shown in FIG. 32, the distal end portion 1421 of the outer shaft 1410 has an implant engagement member 1422 configured to receive the external tool head of an implant such as the external tool head 2185 of the implant 2100 described above and shown in FIG. 33. In this embodiment, the implant engagement member 1422 is hexagon shaped, but other shapes and configuration can alternatively be used.

Intermediate shaft 1430 of the implant insertion/removal tool 1400 includes a proximal end portion 1431 and a distal end portion 1441 (see e.g., FIG. 30). Intermediate shaft 1430 also defines a lumen (not shown) configured to receive the inner shaft 1450 of the implant insertion/removal tool 1400. Distal end portion 1441 of the intermediate shaft 1430 has a threaded portion 1442 configured to be threadedly coupled to the inner surface of the external tool head of an implant such as the inner surface of the external tool head 2185 of the implant 2100.

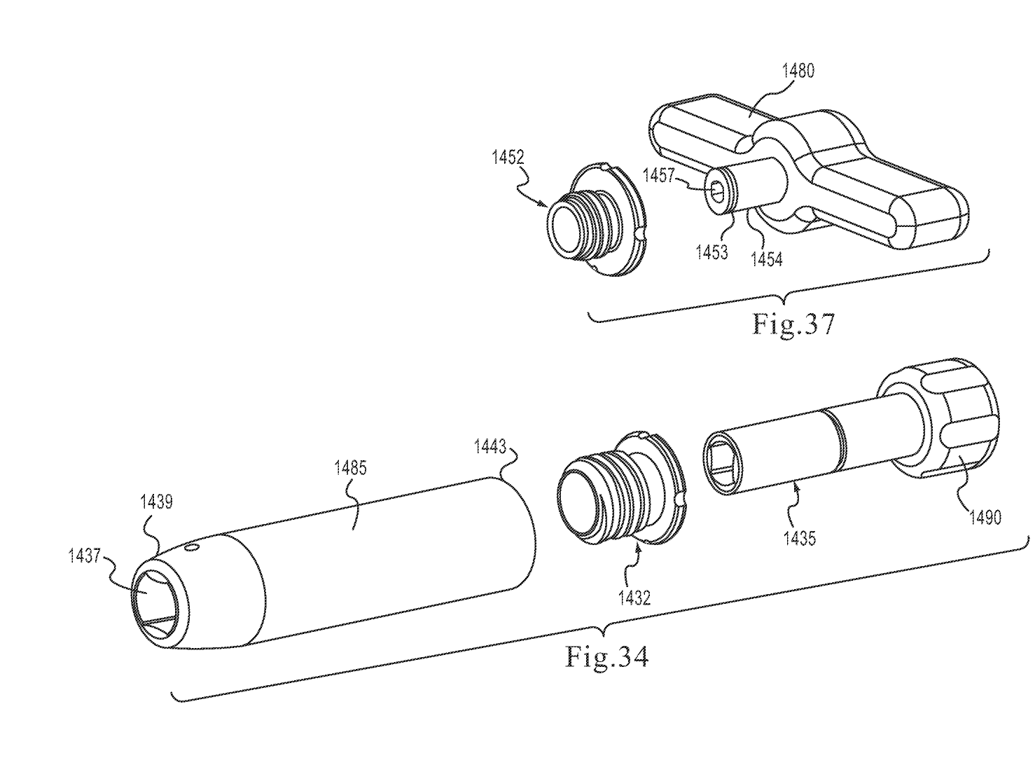

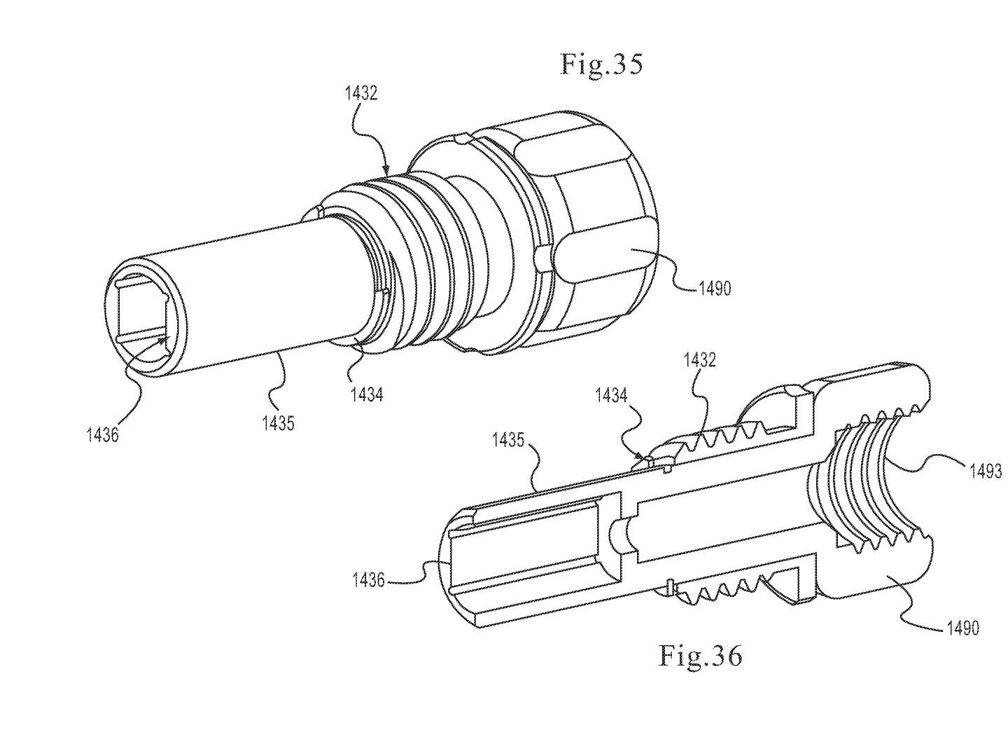

As shown in FIGS. 28 and 34-36, the proximal end portion 1431 of the intermediate shaft 1430 is configured to be received in a keyway 1436 of an elongate portion 1435 of the release knob 1490. As best shown in FIGS. 34-36, a housing coupler 1432 is coupled to the elongate portion 1435 of the release knob 1490 and a retainer 1434, such as an E-ring, retains the housing coupler 1432 on the release knob 1490, while still allowing independent rotational movement between the housing coupler 1432 and the release knob 1490. The elongate portion 1435 is disposed through a proximal end 1443 of the housing 1485. The threads on the housing coupler 1432 are threaded into a threaded portion 1483 (see FIG. 28) within the lumen 1437 of the housing 1485. A central spring 1425 is coupled to the proximal end portion 1431 of the intermediate shaft 1430 to bias the intermediate shaft 1430 distally.

Inner shaft 1450 of the implant insertion/removal tool 1400 includes a proximal end portion 1451 and a distal end portion 1461 (see e.g., FIG. 31). The distal end portion 1461 of the inner shaft 1450 has a drive member 1462 configured to engage the tool head of the drive screw of an implant such as the tool head 2184 of the drive screw 2183 of the implant 2100. The inner shaft 1450 extends through the intermediate shaft 1430, through the release knob 1490, and the proximal end portion 1451 of the inner shaft 1450 is coupled to the actuation handle 1480.

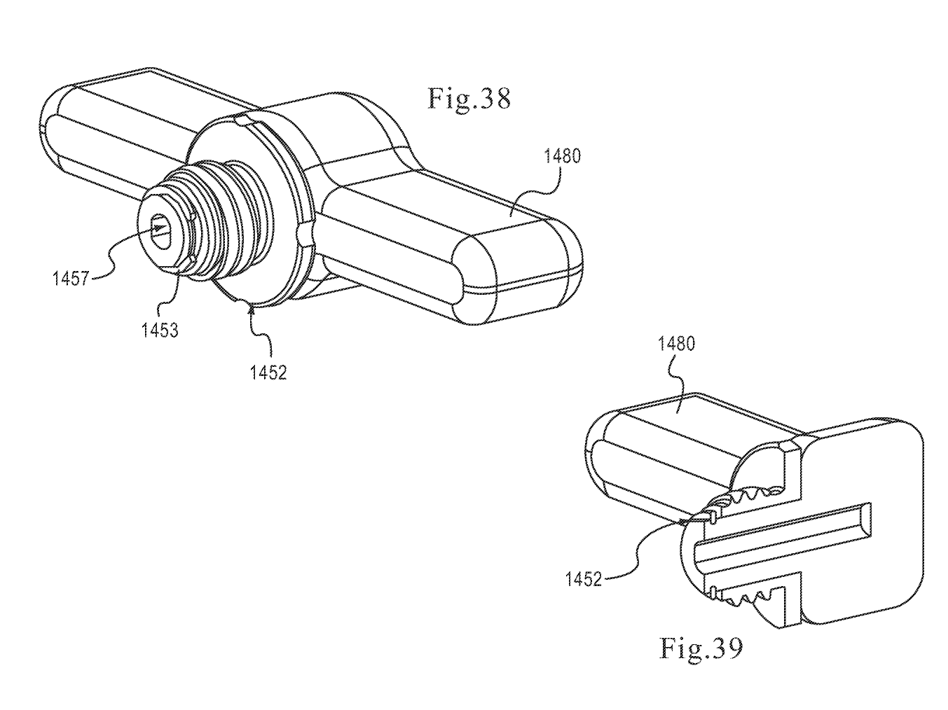

As shown in FIG. 28, the handle 1480 is coupled to a proximal end of the release knob 1490. As shown in FIGS. 37-39, a release knob coupler 1452 couples to a post 1454, and a retainer 1453 is disposed on an end of the post 1454. The retainer 1453 can be, for example, an E-ring configured to retain the release knob coupler 1452 on the post 1454 while still allowing independent movement between the release knob 1490 and the handle 1480 (see FIG. 38). The release knob coupler 1452 is threaded into a threaded portion 1493 of the release knob 1490. The post 1454 defines a keyway 1457 configured to receive the distal end portion 1451 of the inner shaft 1450. A drive spring 1427 (see FIG. 28) is coupled to the proximal end portion 1451 of the inner shaft 1450 to bias the inner shaft 1450 into an extended position in which a distal end of the driver member 1462 extends distally of the intermediate shaft 1430 and the outer shaft 1410. This ensures that the drive member 1462 fits tightly into the tool head (e.g., tool head 2184) of the drive screw (e.g., drive screw 2183).

The implant insertion/removal tool 1400, can be used to percutaneously insert an implant (e.g., implant 2100) into a space in a body such as between adjacent spinous processes or within an intervertebral disc space. The insertion/removal tool 1400 is first coupled to the implant 2100 while the implant 2100 is in a first configuration (e.g., collapsed configuration). The drive member 1462 is inserted through the tool engagement member 2182 (see FIG. 33) such that the drive member 1462 engages the tool head 2184 of the drive screw 2183 and the hexagon-shaped portion of the implant engagement member 1422 engages the hex portion 2185 of the implant 2100. The release knob 1490 is rotated, which rotates the intermediate shaft 1430, and in turn threadedly couples the threaded portion 1442 of the intermediate shaft 1430 to the threaded portion 2190 of the implant 2100.

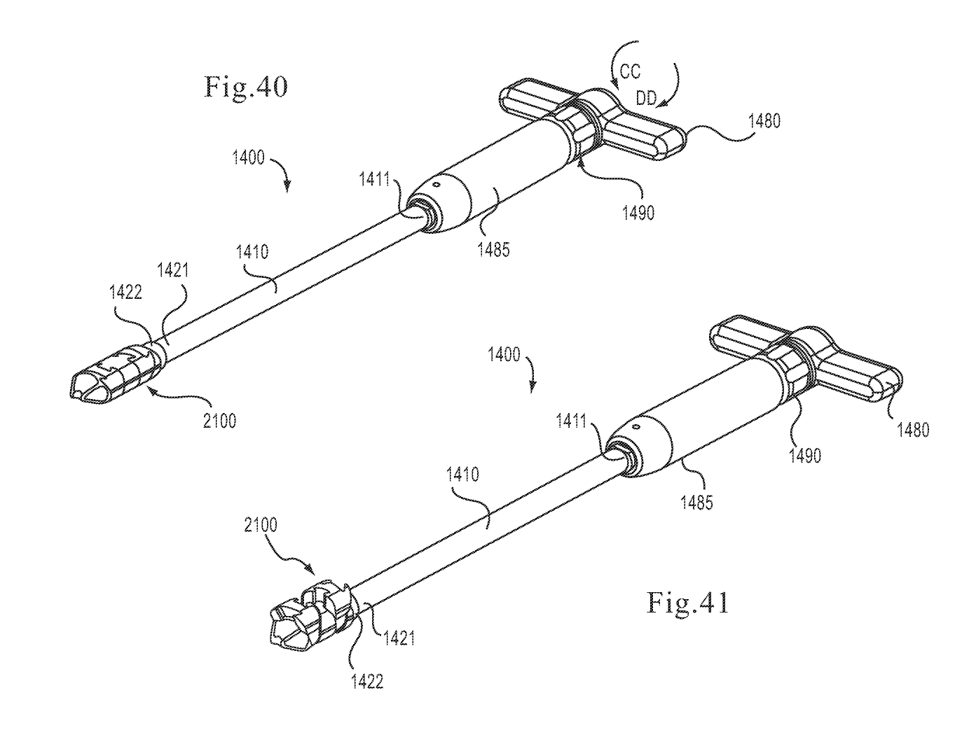

With the insertion/removal tool 1400 attached to the implant 2100, the tool engagement member 2182 prevents the implant 2100 from rotating relative to the insertion/removal tool 1400. In addition, the threaded coupling of the intermediate shaft 1430 to the implant 2100 prevents the implant from moving longitudinally relative to the tool 1400 and also prevents the drive screw 2183 from moving longitudinally. Moreover, as described above when the shaft 1430 of the insertion tool is coupled within the threaded portion 2190 of the implant 2100, movement of the drive screw 2183 along the longitudinal axis relative to the tool engagement member 2182 is limited (i.e., the screw 2183 cannot “back out”). FIG. 40 illustrates the implant 2100 in the first configuration (e.g., collapsed configuration) coupled to the insertion/removal tool 1400.

The insertion/removal tool 1400 can then be used to insert percutaneously the implant into a desired location within a patient's body, such as in a space between adjacent spinous processes. For example, a medical practitioner can insert the implant 2100 percutaneously through a cannula into a body of a patient. Once the implant is in the desired position, the actuation handle 1480 can be rotated as indicated by the arrow CC in FIG. 40 independent of the housing 1485 and the release knob 1490. This in turn rotates the inner shaft 1450 of the insertion/removal tool 1400 and the drive member 1462 of the distal end portion 1461 of the inner shaft 1450. Rotation of the drive member 1462 in turn rotates the drive screw 2184 of the implant 2100 and moves the implant 2100 into a second configuration (e.g., expanded configuration) as shown in FIG. 41.

After actuating the implant 2100 to the second configuration, the release knob 1490 can be rotated in an opposite direction as indicated by the arrow DD in FIG. 40 independent of the housing 1485 and the actuation handle 1480. This causes the intermediate shaft 1430 and the threaded portion 1442 of the intermediate shaft 1430 to rotate in an opposite direction and in turn causes the threaded portion 1442 of the distal end portion 1441 of the intermediate shaft 1430 to be decoupled from the implant 2100. The implant insertion/removal tool 1400 can then be removed from the body while leaving the implant 2100 behind in the body of a patient.

The implant insertion/removal tool 1400 can remove and/or reposition an implant already disposed within the body of a patient. The insertion/removal tool 1400 can be inserted into the patient's body and secured to the implant in the same manner as described above. In some embodiments, a portion of the implant and/or a portion of the insertion/removal tool 1400 can be configured to facilitate the docking of the insertion/removal tool 1400 onto the implant. For example, in some embodiments, the outer surface of the implant and/or a corresponding inner surface of the insertion/removal tool 1400 can include a lead-in chamfer, a tapered portion and/or a beveled edge to facilitate the docking of the insertion tool onto the implant. After the insertion/removal tool 1400 is secured to the implant, the insertion/removal tool 1400 can then be actuated to move the implant to the first configuration (e.g., collapsed configuration). The implant can then be moved to a new location within the patient's body or removed form the patient's body.

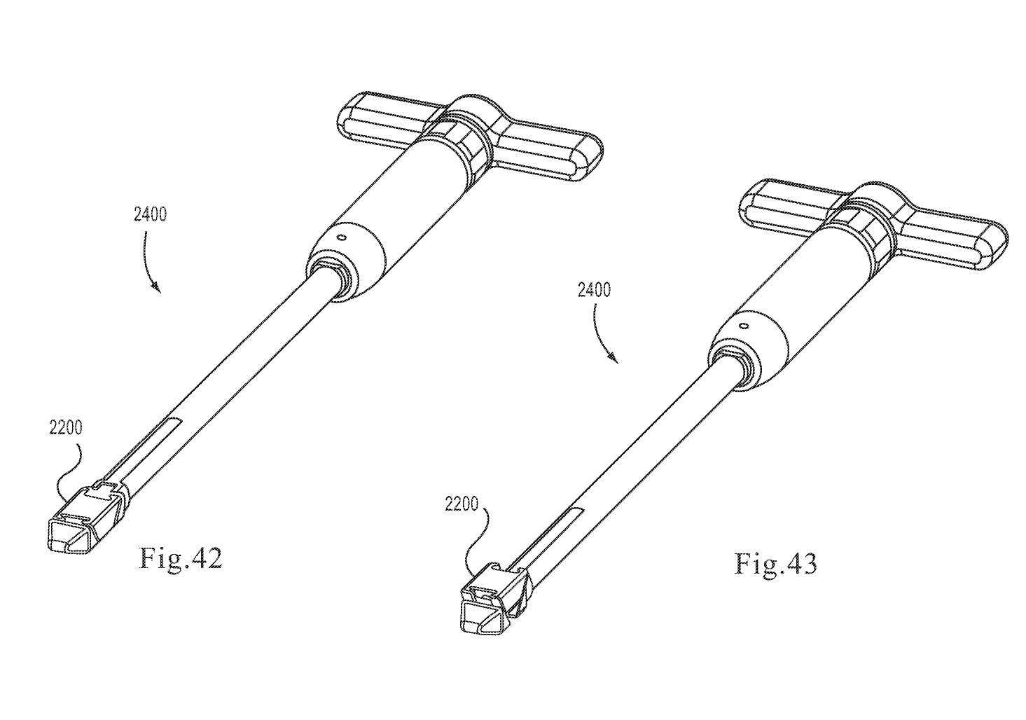

FIGS. 42 and 43 illustrate an implant insertion/removal tool 2400, according to another embodiment. Implant insertion/removal tool 2400 has a similar structure to and can operate in a similar manner as the implant insertion/removal tool 1400. Implant insertion/removal tool 2400 is configured to be used with an implant 2200 configured to be inserted into an intervertebral disc space. FIG. 42 shows the implant 2200 in a first or collapsed configuration and FIG. 43 shows the implant 2200 in a second or expanded configuration. The implant 2200 is described in more detail in U.S. Patent Application Attorney Docket No. KYPH-040/01US 305363-2277, which is incorporated herein by reference in its entirety.

In some embodiments, the implant insertion/removal tool 2400 and the implant 2200 can be used to distract a disc space (not shown) and/or define a void within a vertebra (not shown). In some embodiments, the distal portion of the tool 2400 can be inserted into a vertebra such that the implant 2200 is within the cancellous bone portion of vertebra. The distal end portion of the tool 2400 can be inserted percutaneously via a pedicular approach. After the implant 2200 is disposed within the vertebra, the tool 2400 can be actuated, as described above such that the implant is moved from a collapsed configuration to an expanded configuration. In this manner, the tool 2400 and the implant 2200 can be used to define a void within the cancellous bone. Moreover, in some embodiments, the tool 2400 and the implant 2200 can be used repair a bone defect by moving an endplate of the vertebra. In some embodiments, the tool 2400 can include a measurement device, such as that shown and described above with reference to tool 1300, to provide the user with an indication of the size change of the implant 2200.

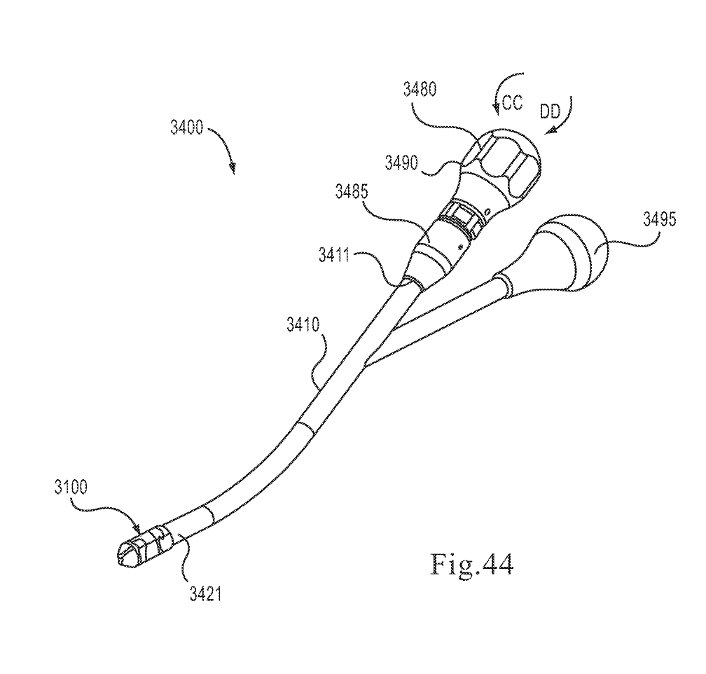

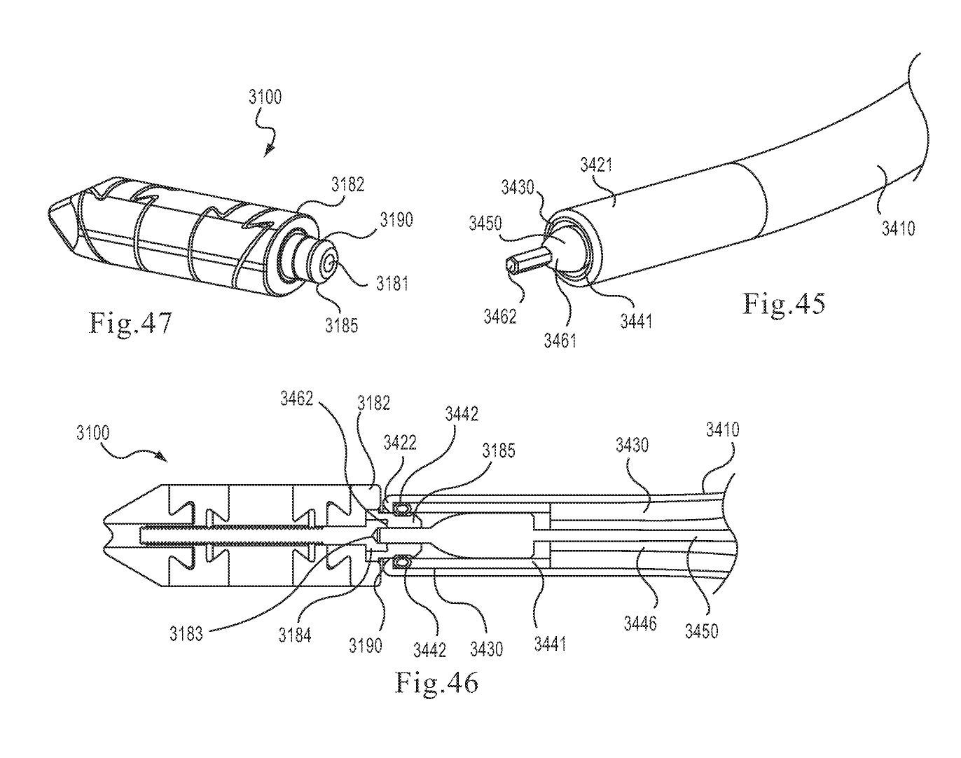

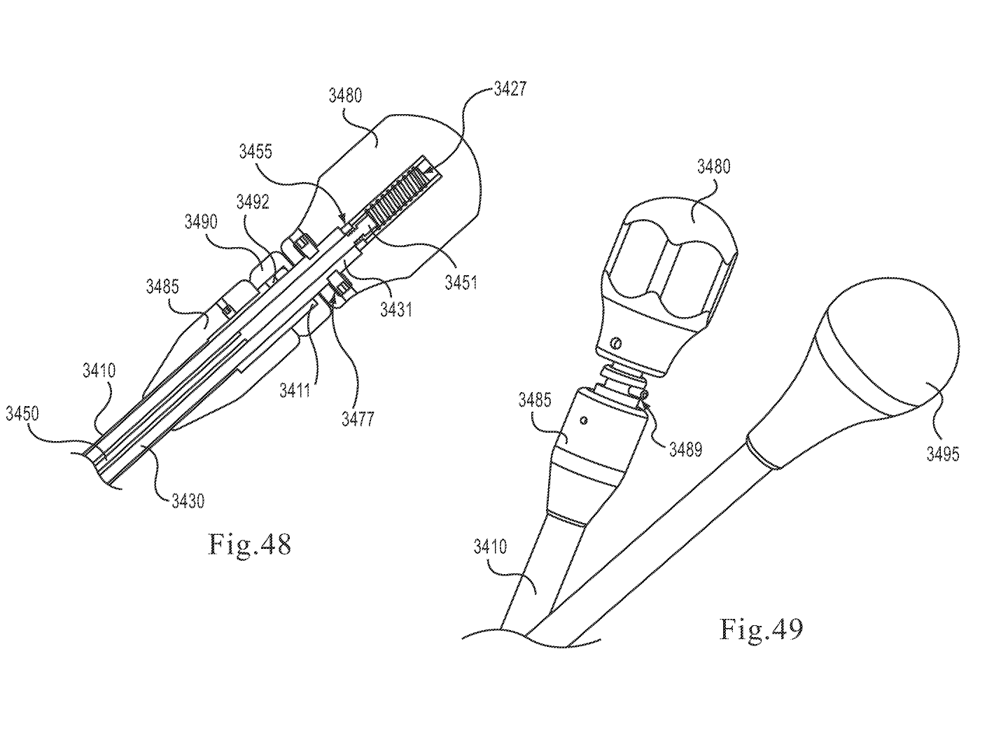

FIGS. 44-54 illustrate an implant insertion/removal tool 3400, according to another embodiment of the invention. The insertion/removal tool 3400 can be used to insert/remove and actuate an implant between a first configuration (e.g., collapsed configuration) and a second configuration (e.g., expanded configuration). FIG. 44 shows the insertion/removal tool 3400 coupled to an implant 3100.

The implant 3100 is configured similar to and can function in a similar manner as the implant 2100 described above. As shown in FIGS. 46 and 47, the implant 3100 includes a tool engagement member 3182 that includes a coupling protrusion 3185. The tool coupling protrusion 3185 is configured to be removably coupled to an insertion tool, such as insertion/removal tool 3400. The implant 3100 also includes a drive screw 3183 that has a tool head 3184. The drive screw 3183 can be actuated to move the implant 3100 between a first configuration and a second configuration. The coupling of the insertion/removal tool 3400 to the implant 3100 is described in more detail below.

The implant insertion/removal tool 3400 (also referred to herein as “insertion/removal tool”) includes an outer shaft 3410, an intermediate shaft 3430, an inner shaft 3450, an actuation handle 3480, a housing 3485, a release knob 3490 and a support handle 3495. The actuation handle 3480 is coupled to the inner shaft 3450 and is configured to rotate the inner shaft 3450 about a centerline of the actuation handle 3480 in a similar manner as described above for insertion/removal tool 1400. The release knob 3490 is coupled to the intermediate shaft 3430 and is configured to move the intermediate shaft 3430 proximally and distally as described in more detail below. The support handle 3495 is offset from the outer shaft 3410 and is used to stabilize the implant insertion/removal tool 3400 during the insertion or removal of an implant.

The outer shaft 3410 of the implant insertion/removal tool 3400 includes a proximal end portion 3411 and a distal end portion 3421 (see e.g., FIGS. 44 and 49). Outer shaft 3410 of the implant insertion/removal tool 3400 also defines a lumen (not shown). The intermediate shaft 3430 of the implant insertion/removal tool 3400 is configured to be disposed within the lumen defined by the outer shaft 3410. The proximal end portion 3411 of the outer shaft 3410 is coupled to the housing 3485 and the release knob 3490. The distal end portion 3421 of the outer shaft 3410 includes an implant engagement portion 3422 configured to receive an external tool head of an implant, such as the external tool head 3185 of the implant 3100 shown in FIGS. 46 and 47.

The intermediate shaft 3430 of the implant insertion/removal tool 3400 includes a proximal end portion 3431 and a distal end portion 3441 (see e.g., FIGS. 46, 48 and 50) and defines a lumen 3446 (see FIG. 46). The inner shaft 3450 of the implant insertion/removal tool 3400 is configured to be disposed within the lumen 3446 defined by the intermediate shaft 3430. The proximal end portion 3431 of the intermediate shaft 3430 is coupled to the release knob 3490 of the implant insertion/removal tool 3400. A spring-loaded quick connect fitting 3442 is disposed within the outer shaft 3410 at a distal end of the intermediate shaft 3430. The spring-loaded quick connect fitting 3442 can be, for example, a snap-ring or spring coil. The spring-loaded quick connect fitting 3442 can be compressed between an external tool head of an implant and the distal end portion 3441 of the intermediate shaft 3430

For example, the tool coupling protrusion 3185 of the implant 3100 includes a groove or detent 3190 configured to receive the quick connect fitting 3442 of the insertion/removal tool 3400. The intermediate shaft 3430 of the insertion/removal tool 3400 can be moved proximally and distally to produce more or less interference between the implant 3100 and the fitting 3442. Actuation of the intermediate shaft 3430 by rotating the release knob 3490 is described in more detail below. When the intermediate shaft 3430 is moved distally such that more interference is produced, the fitting 3443 produces a lock between the implant 3100 and the insertion/removal tool 3400. Retracting the intermediate shaft 3430 (e.g., moving it proximally) allows the intermediate shaft 3430 to detach from the implant 3100. For example, a user can apply a slight pulling force on the insertion/removal tool 3400. Thus, the fitting 3442 and the groove 3190 can collectively form an interference fit such that both axial and rotational movement of the implant 3100 relative to the insertion tool 3400 is limited or prevented.

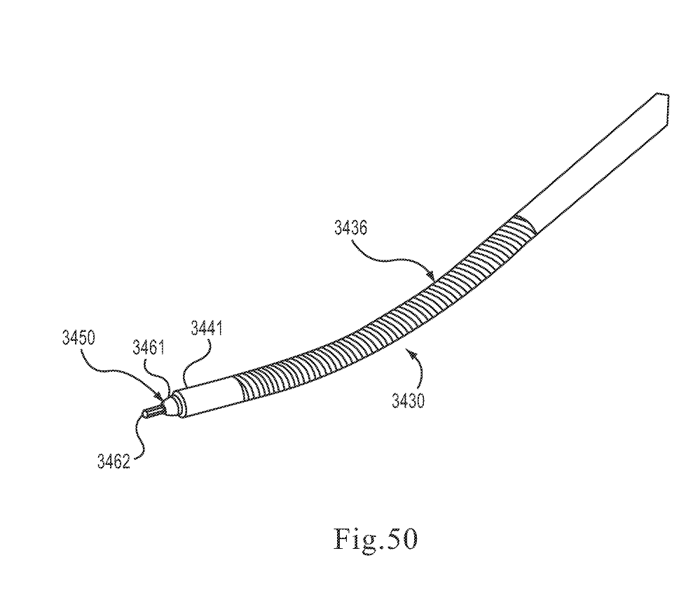

As shown in FIG. 50, the intermediate shaft 3430 includes a coil portion 3436 that is bendable yet torsionally and compressively stiff. The coil portion 3436 allows a compression load to be applied to the fitting 3442 while being maneuverable with the outer shaft 3410 and permitting rotation of the inner shaft 3450 within the lumen 3446 of the intermediate shaft 3430. The proximal end portion 3431 and the distal end portion 3441 can be formed with, for example, cannulated tubing, which can be attached to the coil portion 3436. The coil portion 3436 can be various lengths of the intermediate shaft 3430. In some embodiments, a coil portion is not included.

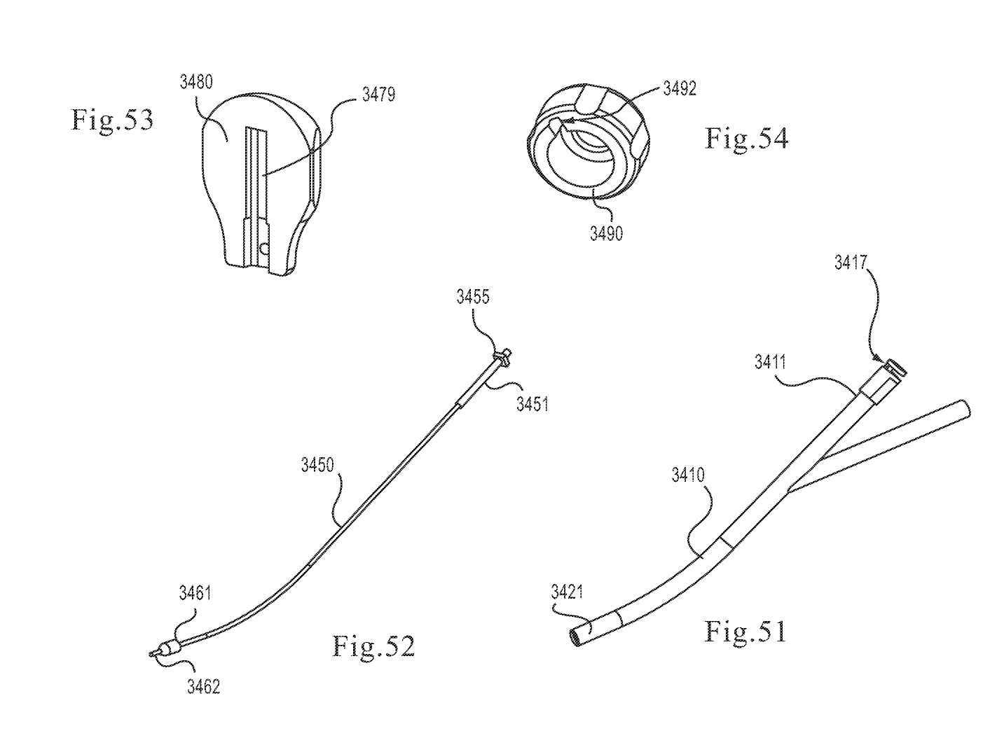

As shown in FIG. 49, a pin 3489 is attached to the proximal end portion 3431 of the intermediate shaft 3430. The pin 3489 is keyed into a slot 3492 of the release knob 3490 shown in FIG. 54. During actuation of the intermediate shaft 3430, the pin 3489 rides on a cam feature 3417 on the outer shaft 3410 shown in FIG. 51. The cam feature 3417 drives the intermediate shaft 3430 proximally or distally as the release knob 3490 is rotated allowing the insertion/removal tool 3400 to release or lock onto an implant.

The inner shaft 3450 of the implant insertion/removal tool 3400 includes a proximal end portion 3451 and a distal end portion 3461 (see e.g., FIGS. 46, 48 and 52). The distal end portion 3461 of the inner shaft 3450 includes a drive member 3462 configured to engage the tool head of the drive screw of an implant such as the tool head 3184 of the drive screw 3183 of the implant 3100 shown in FIGS. 46 and 47.

The proximal end portion 3451 of the inner shaft 3450 is coupled to the actuation handle 3480 of the implant insertion/removal tool 3400. The proximal end portion 3451 inner shaft 3450 include a flange 3455 (shown in FIG. 52) configured to be keyed into a slot 3479 of the actuation handle 3480 shown in FIG. 51. A drive spring 3427 is also disposed within the slot 3479 of the handle 3480 and biases the inner shaft 3450 distally to ensure the drive member 3462 fits tightly into the tool head of the drive screw. Screws 3477 coupled to the handle 3480 are keyed into the outer shaft 3410 to restrict axial movement of the handle 3480, but allow rotational movement. Thus, the handle 3480 can be rotated to actuate rotational movement of the inner shaft 3450.

As described above for implant insertion/removal tool 1400, the implant insertion/removal tool 3400 can be coupled to an implant and used to insert/remove the implant within a body of a patient and can also be used to actuate the implant between a first configuration and a second configuration. For example, the insertion/removal tool 3400 can be used to percutaneously insert an implant in a first configuration into a space between adjacent spinous processes or within an intervertebral disc space.

To couple the insertion/removal tool 3400 to an implant, such as the implant 3100, the driver member 3462 of the inner shaft 3450 is inserted through an opening 3181 of the tool engagement portion 3182 of the implant 3100 such that the driver member 3462 engages the tool head 3483 of the drive screw 3484. As the driver member 3462 is being inserted, the fitting 3442 can be moved into the groove 3190 of the tool engagement portion 3182. The release knob 3490 can be rotated to move the intermediate shaft 3420 distally to produce interference with the fitting 3442 and lock the insertion/removal tool 3400 to the implant 3100. With the implant 3100 in a first configuration (e.g., collapsed), the implant 3100 can be inserted into a desired location within a patient's body.

Once the implant is in place, the actuation handle 3480 can be rotated as indicated by the arrow CC in FIG. 44. This in turn rotates the inner shaft 3450 of the insertion/removal tool 3400 and thus the drive member 3462 of the distal end portion 3461 of the inner shaft 3450. Rotation of the drive member 3462 of the distal end portion 3461 of the inner shaft 3450 in turn rotates the drive screw 3184 of the implant 3100 and moves the implant 3100 to the second configuration (not shown).

After the implant 3100 has been moved to the second configuration (e.g., expanded configuration), the release knob 3490 can be rotated in an opposite direction as indicated by the arrow DD in FIG. 44. This causes the intermediate shaft 3430 to translate in a proximal direction. The translation releases the interference between the intermediate shaft 3430 and quick connect fitting 3442 and allows the insertion/removal tool 3400 to be detached from the implant 3100. The implant insertion/removal tool 3400 can then be removed from the body while leaving the implant 3100 behind in the body of the patient.

The implant insertion/removal tool 3400 can also be used to remove and/or reposition an implant. The insertion/removal tool 3400 can be secured to an implant while the implant is still disposed within the patient's body in the same manner as described above. With the implant secured to the insertion/removal tool 3400, the implant can be moved to its first configuration (e.g., collapsed configuration) by rotating the actuation handle 3480 of the implant insertion/removal tool 3400 as indicated by the arrow CC in FIG. 44. The implant, in its first configuration, can then be removed and/or repositioned.

The various implants, insertion/removal tools, and dilation devices described herein can be constructed with various biocompatible materials such as, for example, titanium, titanium alloyed, surgical steel, biocompatible metal alloys, stainless steel, plastic, polyetheretherketone (PEEK), carbon fiber, ultra-high molecular weight (UHMW) polyethylene, biocompatible polymeric materials, etc. The material of one portion of a tool or implant can be different than another portion.

While various embodiments of the invention have been described above, it should be understood that they have been presented by way of example only, and not limitation. Where methods and steps described above indicate certain events occurring in certain order, ordering of certain steps may be modified. Additionally, certain of the steps may be performed concurrently in a parallel process when possible, as well as performed sequentially as described above. While specific embodiments have been described, it will be understood that various changes in form and details may be made.

Although the insertion/removal tools described herein were described in connection with specific embodiments of spinal implants, such as implants configured to be disposed within an intervertebral disc space or in a space between adjacent interspinous processes, and the insertion/removal tools can be used with other types of implants having various configurations. Moreover, although the insertion/removal tools (e.g., 1400, 2400, 3400) have been described as being used to insert and/or remove and actuate and implant, the insertion/removal tools can also be used to insert and actuate a dilation device (e.g., dilation head 3110).

In addition, although the dilation tools described herein were described as having a particular embodiment of a dilation head, other types of dilation heads can alternatively be incorporated. For example, different embodiments of an expandable dilation head can be configured to be inserted into a patient's body and actuated using the actuation portion of the dilation tools described herein. Likewise, the dilation head (e.g., 1310) can be configured to be actuated using a different embodiment of an actuation device. For example, the dilation head 1310 can be configured to be coupled to, and actuated with, an insertion/removal tool (e.g., 1400, 3400) as described herein. In another example, the various spinal implants described herein can also be configured to be actuated using an actuation portion as described for dilation tool 1300.

Thus, although various embodiments have been described as having particular features and/or combinations of components, other embodiments are possible having a combination of any features and/or components from any of the embodiments (e.g., dilation tool 1300, insertion/removal tools 1400, 2400, 3400) where appropriate. For example, the various shafts of the insertion/removal tools can include different types of coupling features to couple the insertion/removal tool to an implant. In another example, the driver member can have a variety of different shapes, sizes and configurations configured to matingly engage a drive mechanism of an implant not specifically described.

What is claimed is:

1. An apparatus, comprising:
an elongate member;
a measurement tool coupled to a distal end portion of the elongate member, a size of the measurement tool configured to change by a first amount when the measurement tool is moved between a first configuration and a second configuration;
an actuator coupled to a proximal end portion of the elongate member, the actuator configured to rotate about an axis substantially parallel to at least a portion of a center line of the elongate member to move the measurement tool between the first configuration and the second configuration; and
a size indicator disposed at a proximal end portion of the elongate member, the size indicator configured to move axially relative to the elongate member by a second amount when the measurement tool is moved between the first configuration and the second configuration.

2. The apparatus of claim 1, wherein at least a portion of the center line of the elongate member is non-linear.

3. The apparatus of claim 1, wherein:
the elongate member has a first shaft and a second shaft, at least a portion of the second shaft being movably disposed within first shaft; and
the actuator is configured to rotate the second shaft relative to the first shaft to move the measurement tool between the first configuration and the second configuration.

4. The apparatus of claim 1, wherein the measurement tool is configured to be disposed within a space between adjacent spinous processes.

5. The apparatus of claim 1, wherein the measurement tool is configured to distract adjacent spinous processes.

6. The apparatus of claim 1, wherein:
the actuator is a proximal actuator; and
the measurement tool includes:
a spacer having a first spacer member and a second spacer member, the first spacer member configured to move relative to the second spacer member by the first amount when the measurement tool is moved between the first configuration and the second configuration; and
a distal actuator having a first actuator member and a second actuator member coupled to the first actuator member, the first actuator member being matingly and movably coupled to the first spacer member and the second spacer member, the second actuator member being matingly and movably coupled to the first spacer member and the second spacer member, the distal actuator configured to move the first spacer member relative to the second spacer member when the proximal actuator is rotated.

7. The apparatus of claim 1, wherein the measurement tool includes a first spacer member and a second spacer member, a substantially planar surface of the first spacer member configured to move relative to a substantially planar surface of the second spacer member by the first amount when the measurement tool is moved between the first configuration and the second configuration.

8. The apparatus of claim 1, wherein the size of the measurement tool configured to change within a range from approximately 8 millimeters to approximately 16 millimeters.

9. The apparatus of claim 1, wherein the size of the measurement tool configured to change by approximately 2 millimeters to approximately 4 millimeters.

10. The apparatus of claim 1, further comprising a locking member configured to be removably coupled to the elongate member, the locking member configured to limit rotation of the actuator relative to the elongate member.

11. An apparatus, comprising:
an elongate member, a center line of the elongate member being non-linear, the elongate member having a first shaft and a second shaft, at least a portion of the second shaft being movably disposed within first shaft;
a measurement tool coupled to a distal end portion of the elongate member, a size of the measurement tool configured to change when the measurement tool is moved between a first configuration and a second configuration;
an actuator configured to rotate the second shaft relative to the first shaft to move the measurement tool between the first configuration and the second configuration; and
a size indicator configured to indicate the change in the size of the measurement tool when the measurement tool is moved between the first configuration and the second configuration.

12. The apparatus of claim 11, wherein:
the actuator is a proximal actuator; and
the measurement tool includes:
a spacer having a first spacer member and a second spacer member, the first spacer member configured to move relative to the second spacer member when the measurement tool is moved between the first configuration and the second configuration; and
a distal actuator having a first actuator member and a second actuator member coupled to the first actuator member, the first actuator member being matingly and movably coupled to the first spacer member and the second spacer member, the second actuator member being matingly and movably coupled to the first spacer member and the second spacer member, the distal actuator configured to move the first spacer member relative to the second spacer member when the proximal actuator is rotated.

13. The apparatus of claim 11, wherein:
the actuator is a proximal actuator; and
the measurement tool includes:
a spacer having a first spacer member and a second spacer member, the first spacer member configured to move relative to the second spacer member when the measurement tool is moved between the first configuration and the second configuration, the first spacer member defining a groove having a trapezoidal cross-sectional shape, the second spacer member defining a groove having a trapezoidal cross-sectional shape; and
a distal actuator having including a first protrusion and a second protrusion, each of the first protrusion and the second protrusion of the distal actuator having a trapezoidal cross-sectional shape, the first protrusion of the distal actuator member received within the groove of the first spacer member such that the first spacer member is matingly and movably coupled to the distal actuator, the second protrusion of the distal actuator received within the groove of the second spacer member such that the second spacer member is matingly and movably coupled to the distal actuator, the distal actuator configured to move the first spacer member relative to the second spacer member when the proximal actuator is rotated.

14. The apparatus of claim 11, wherein the measurement tool includes a first spacer member and a second spacer member, a substantially planar surface of the first spacer member configured to move relative to a substantially planar surface of the second spacer member when the measurement tool is moved between the first configuration and the second configuration.

15. The apparatus of claim 11, wherein the size indicator is configured to provide at least one of a quantitative indication or a qualitative indication of the size of the measurement tool.

16. The apparatus of claim 11, wherein the size indicator is coupled to the actuator such that rotation of the size indicator relative to the actuator is limited, the size indicator is coupled to the actuator such that the size indicator can move relative to the actuator along the center line of the elongate member.

17. The apparatus of claim 11, further comprising a locking member configured to be removably coupled to the elongate member, the locking member configured to limit rotation of the actuator relative to the elongate member.

18. An apparatus, comprising:
an elongate member;
a measurement tool coupled to a distal end portion of the elongate member, a size of the measurement tool configured to change when the measurement tool is moved between a first configuration and a second configuration the measurement tool having:
a spacer having a first spacer member and a second spacer member, the first spacer member configured to move relative to the second spacer member when the measurement tool is moved between the first configuration and the second configuration, and
a distal actuator having a first actuator surface and a second actuator surface, the first actuator surface being matingly and movably coupled to the first spacer member, the second actuator surface being matingly and movably coupled to the second spacer member; and
a proximal actuator coupled to a proximal end portion of the elongate member, the actuator configured to rotate about an axis substantially parallel to at least a portion of a center line of the elongate member to move the distal actuator, the distal actuator configured to move the first spacer member relative to the second spacer.

19. The apparatus of claim 18, wherein at least a portion of the center line of the elongate member is non-linear.

20. The apparatus of claim 18, wherein:
the elongate member has a first shaft and a second shaft, at least a portion of the second shaft being movably disposed within first shaft; and
the proximal actuator is configured to rotate the second shaft relative to the first shaft to move the measurement tool between the first configuration and the second configuration.

21. The apparatus of claim 18, wherein:
the first spacer member defines a groove having a trapezoidal cross-sectional shape;
the second spacer member defines a groove having a trapezoidal cross-sectional shape;
the first actuator surface of the distal actuator has a protrusion having trapezoidal cross-sectional shape received within the groove of the first spacer member such that the first spacer member is matingly and movably coupled to the distal actuator; and
the second actuator surface of the distal actuator has a protrusion having trapezoidal cross-sectional shape received within the groove of the second spacer member such that the second spacer member is matingly and movably coupled to the distal actuator

22. The apparatus of claim 18, further comprising:
a size indicator configured to indicate the change in the size of the measurement tool when the measurement tool is moved between the first configuration and the second configuration.
